(19) United States
(12) Patent Application Publication
(10) Pub. No.: US 2010/0216854 A1
(43) Pub. Date: Aug. 26, 2010

(54) 1,3 AND 1,3,5 SUBSTITUTED IMIDAZOLES AS ANTIHYPERTENSIVES

(75) Inventors: John Matsoukas, Patras (GR); Charalambos Gavras, Thessaloniki (GR); Dimitrios Vlaxakos, Athens (GR); Michael Maragoudakis, Patras (GR)

Correspondence Address: Brinks Hofer Gilson & Lione/Ann Arbor, 524 South Main Street, Suite 200, Ann Arbor, MI, 48104, US

(73) Assignee: Eldrug S.A., Patras (GR)

(21) Appl. No.: 12/671,242

(22) PCT Filed: Jul. 25, 2008
(86) PCT No.: PCT/IB08/02796

§ 371 Date: Apr. 30, 2010

(30) Foreign Application Priority Data
Aug. 1, 2007 (GB) 0714995.8
Oct. 24, 2007 (GR) 20070100650

(51) Int. Cl. A61K 31/4178; C07D 233/64; A61K 31/417; A61P 9/12; C07D 403/14

(52) U.S. Cl. 514/381; 548/336.1; 514/400; 548/254

(57) ABSTRACT

The present invention provides novel 1,5 and 1,3,5-substituted imidazole compounds of formulas (I), (IIa), (IIIb) in hydrophilic or lipophilic form, which are useful as angiotensin II AT1 receptor antagonists with sympathetic suppressant properties. In particular, the invention provides pharmaceutical compositions containing the pharmacophoric groups of Losartan and Clonidine as well compounds, processes and intermediates for preparing compounds and their use in methods of treating hypertension and cardiovascular diseases through Renin Angiotensin System (RAS) and Sympathetic System (SS). Alkylated histamine based double action Saltans are lipophilic and can act transdermally.

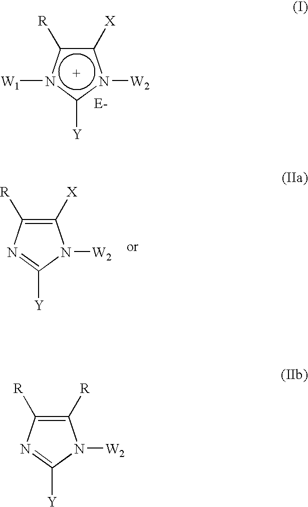

This invention provides novel 1,5- and 1,3,5-substituted imidazole Angiotensin II AT1 Receptor Antagonists based on histamine in hydrophilic and lipophilic forms. The compounds of the invention have sympathetic suppressant properties and are thus useful in the treatment of certain cardiovascular diseases.

### BACKGROUND TO THE INVENTION

(A) Angiotensin II Receptor Antagonists

Angiotensin II is an octapeptide hormone (Asp-Arg-Val-Tyr-Ile-His-Pro-Phe) which is a powerful arterial vasoconstrictor that exerts its action by interacting with specific receptors present on cell membranes. Blockade of the actions of angiotensin II using angiotensin receptor antagonists is useful for the treatment of hypertension and congestive heart failure and other cardiovascular and related diseases such as diabetic nephropathy. Pioneering work based on modifications of the peptide structure of ANG II led to potent modified peptides (Sarilesin, Saralasin, Sarmesin) that showed potent and selective in vitro ANG II receptor antagonism. However, the action of such agents in vivo was severely diminished by their rapid metabolism to inactive compounds. Thus, the search was on to identify and develop a non-peptide ANG II receptor antagonist that was both resistant to the metabolic deactivation of peptide antagonists and selective for the ANG II receptor.

In 1982, Takeda (Japan) filed a patent application disclosing the discovery of non-peptide ANG II receptor antagonists. The activity of these initial compounds was low but showed good selectivity. In subsequent years, much detailed knowledge was obtained through work on modified peptides and DuPont engaged in extensive studies to exploit Takeda's early lead. These efforts were rewarded with the development of DuP753 (Losartan), which is now used to treat various hypertensive conditions. The antihypertensive activity of Losartan is largely due to a long-acting metabolite (EXP 3174) which is produced in vivo as a result of the conversion of hydroxymethyl to carboxylate, providing a negative charge required for affinity. Likewise, valsartan (CGP 48933) is a potent angiotensin receptor antagonist containing a carboxylate group analogous to that in EXP 3174. Indeed, a common feature of EXP 3174, CGP 48933 and another angiotensin mimetic SK 108566, is the presence of two acid groups spaced at similar distances on various aromatic templates.

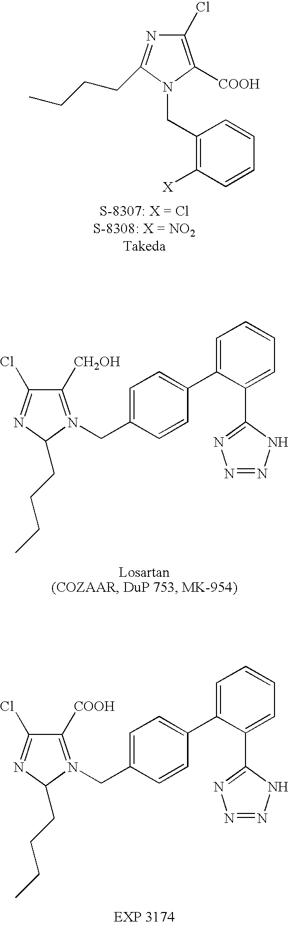

Many other companies have sought to develop their own angiotensin mimetics in a bid to compete for a share in the huge worldwide market for antihypertensive drugs. From these molecules seven antagonists are already in the market; Valsartan was the second molecule after Losartan to reach the market, while Irbersartan, Eprosartan, Candesartan, Telmisartan, Tasosartan and Olmesartan 10 followed. These are collectively known as “Sartans”.

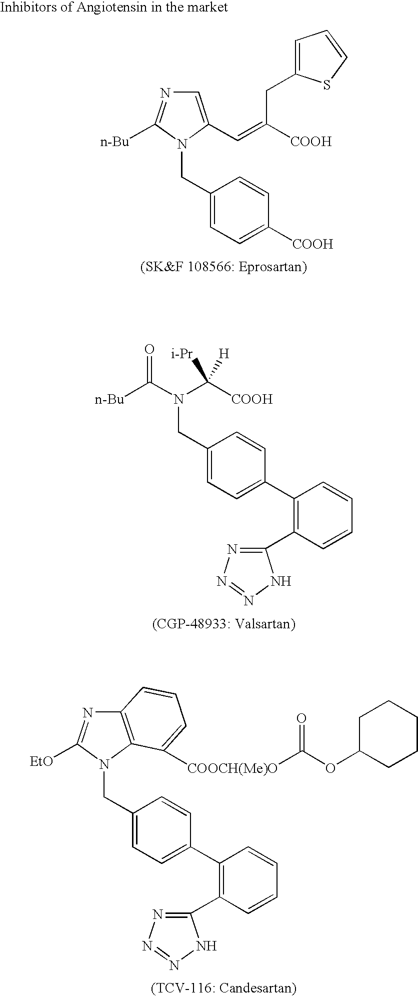

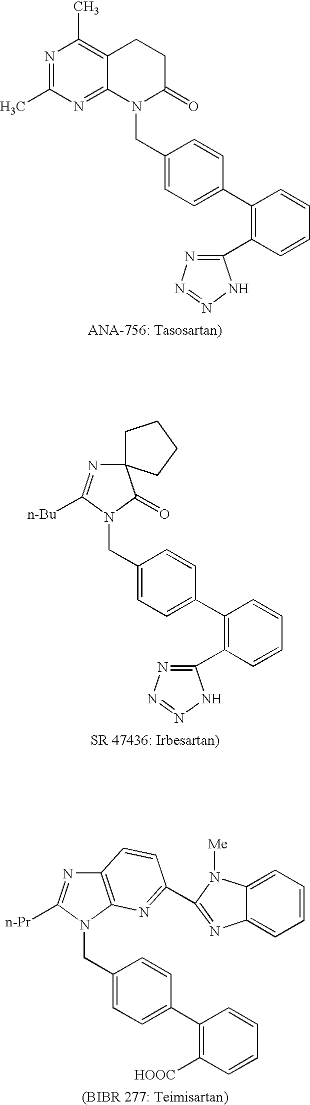

With the exception of Eprosartan, the majority of the above non-peptide antagonists are based on modifications to one or more fragments of Losartan. Thus, there are a number of structural similarities between the compounds on the market.
(a) they generally have a biphenyl scaffold;
(b) the first phenyl (“spacer”) is connected with a nitrogen heterocycle and the second phenyl (“terminal”) with an acidic group such as carboxylic group, tetrazole, sulfonylurea, triflamide or substituted sulfonamide;
(c) most heterocycle rings attached to biphenyl tetrazole (BPT) possess adjacent groups like carboxy groups, basic nitrogen moieties or lactam oxygens that allow hydrogen-bonding to the corresponding acceptor;
(d) all molecules possess an alkyl chain attached to heterocycle; these alkyl chains are believed to fit a lipophilic pocket in the AT1 receptor.

Novel Losartan Analogues and Advanced Synthetic Procedures

For many years, the present applicant has been involved with the study of the pharmacophoric groups of angiotensin II and their spatial relationship when angiotensin II fits into the anhydrous environment of the AT1 receptor (Mavromoustakos et al, J. Med. Chem., 1999). Based on this information, we have designed and developed a number of peptide and non-peptide mimetics with proven ability to decrease blood pressure in our model of angiotensin II-induced hypertension in experimental animals (Vlahakos, Matsoukas et al, LIPS, 1996). These other similar molecules can be effectively used as drugs, since they can be given orally, have good bioavailability, can block AT1 receptors for a long time and are well tolerated with minimal side effects. Experimental and clinical data suggest that an association exists between RAS activation and enhanced erythropoiesis in patients with heart failure, kidney transplantation, renovascular hypertension or on chronic hemodialysis. In a recent publication (Vlahakos et al, Am. J. Med., 1999) we expanded these observations to patients with chronic obstructive pulmonary disease (COPD) and showed that RAS activation plays a fundamental role in the pathogenesis of secondary erythrocytosis. Accordingly, we have successfully used losartan, an AT1 antagonist, to normalize hematocrit in polycythemic COPD patients, an effect that obviates the need for therapeutic phlebotomy (Kosmas, Vlahakos et al, Chest, 1999). ACE inhibitors were deliberately avoided in this study due to the possibility of bradykinin-mediated respiratory side effects in patients with severe pre-existing pulmonary disease.

Our group has been engaged in the synthesis of Angiotensin II receptor antagonists in which: (1) the —CH₂OH and butyl groups are reversed in the imidazole ring compared to Losartan; (2) the tetrazole group is protected by trityl moieties or benzyl derivatives.

Lengthy procedures are usually required to obtain the final product. In certain cases low yields and the formation of stereoisomers increase the overall cost of synthesis. An example is given in the synthesis of potent Losartan analogues described by Wahhab et al in Drug Research, 43, 11, 1993.

Recent work has greatly improved these lead structures and the synthetic procedure has been shortened to a five step regioselective, high yielding sequence suitable for large scale production.

(B) Angiotensin II Receptor Antagonists with Sympathetic Suppressant Properties

Maintenance of abnormally elevated peripheral vascular resistance in hypertension and Chronic Heart Failure (CHF) is due in part to Angiotensin II (ANG II) and to the sympathetic nervous system (SNS). Over the past 25 years several approaches have been employed to inhibit the formation or the activities (via receptor blockade) of either the Ang II or the SNS in order to treat hypertension and/or CHF.

Sympathetic suppression has been attempted by various means over recent years. In most cases, it was effected by means of selective peripheral α and/or β adrenergic receptor blockade, with generally mixed results. Guanidine and analogues have been used as therapeutic agents.

Both of these treatments, separately and in combination, have been used in the past for the treatment of hypertension and/or chronic congestive heart failure (CHF). Combinations of the two treatments appear to be superior to their separate use.

To maintain effective perfusion of the body's organs, the cardiovascular system must meticulously regulate arterial pressure. It does this by continuously altering cardiac output and/or systemic vascular resistance. Preservation of adequate perfusion pressure requires maintenance of appropriate resistance to blood flow by the arterial vasculature. In the systemic vasculature, the major factor controlling vascular resistance is smooth muscle tone, which helps regulate the most important determinant of resistance to flow, the cross-sectional area of a vessel. Abnormalities in this lead to increased arterial blood pressure, a disease known as hypertension.

Hypertension is a common disease and a known risk factor for ischemic heart disease, stroke, peripheral vascular disease, retinopathy and renal failure. Lowering the blood pressure has been proven to reduce mortality and morbidity. However, many hypertensive patients do not achieve adequate blood pressure control. In addition, the goal of treatment should not only seek to lower blood pressure, but also reverse and delay organ damage, such as ventricular and vascular hypertrophy and stiffness, proteinuria etc.

There are two major neurohormonal systems that regulate cardiovascular function, including smooth muscle tone: the renin-angiotensin system (RAS) and the sympathetic nervous system (SNS).

The Renin-Angiotensin System (RAS)

The rennin-angiotensin system is involved in blood pressure regulation and is implicated in the development of hypertension in the vast majority of patients. In addition, it is pathogenetically associated with cardiovascular growth and remodeling. Blocking RAS by angiotensin converting enzyme inhibitors (ACEi) has been a significant advance in cardiovascular therapy and has been shown to reduce cardiovascular morbidity and mortality by ˜30%. Limitations of therapy with ACEi include dry cough and angioedema. Of note, plasma levels of angiotensin II (AII) after prolonged administration of ACEi tend to return towards normal, probably because of the reactive rise of renin and AI levels, which form AII in tissues by alternative pathways, such as cardiac chymase. The pharmacologic effects of AII are mediated through specific cell receptors. There are two major subtypes of the AII receptor, designated as AT1 and AT2. The AT1 receptor is G-protein coupled and mediates most of the known physiological effects of AII, including the maintenance of blood pressure. Although peptide analogues of AII inhibit the action of AII by competitively binding to the receptor, their application as clinical agents is limited due to their short duration of action, poor bioavailability and partial agonist activity. Thus, a new class of non-peptide AII receptor antagonists has been developed and found in clinical trials to be effective and very well tolerated. This is an important issue because by improving compliance a much higher percentage of hypertensive patients can achieve good blood pressure control, whilst decreasing the risk of cardiovascular and renal complications.

The Sympathetic Nervous System (SNS)

The sympathetic nervous system comprises the autonomic outflow from the thoracic and high lumbar segments of the spinal cord. There are two major components involved in SNS function: vasomotor neurons, which regulate vascular resistance, and lumbosacral neurons, which modulate lower urinary tract outlet resistance.

The circulatory system. The inner layer of the blood vessel wall comprises the endothelium, which is now known to be more than an inert anatomic barrier through which blood flows as though through a tube. Instead, the endothelium is an important physiologic organ that is also innervated, like smooth muscle, by the SNS.

Almost all vasomotor nerves are adrenergic. Two types of adrenergic receptors (adrenoceptors), alpha (α) and beta (β), are found in the vasculature. These are distributed in two anatomic areas. In the heart, β₁-adrenoceptors predominate and stimulate the rate and force of cardiac contractions. The α-adrenoceptors predominate in the innervation of the vascular smooth muscle and also in the lower urinary tract. Although the precise roles for each of these adrenoceptor subtypes in the regulation of blood pressure are not completely defined, it is known that these adrenoceptors actively participate in the regulation of the vascular tone, either directly or indirectly (through the release of nitric oxide). A number of sympathetic abnormalities, most notably an increased adrenergic nervous system activity, have been identified as potential causes of high blood pressure.

Alpha 2 adrenergic agonists. α-receptors are a part of the sympathetic nervous system. In the brain, drugs that stimulate α type 2 receptors decrease the sympathetic nervous system activity. Although they are effective at lowering blood pressure, they may produce drowsiness, a feeling of tiredness, and sometimes depression. These drugs include methyldopa and clonidine.

Alpha 1 adrenergic blockers. α-receptors are a part of the sympathetic nervous system. In the blood vessels, alpha type 1 receptors cause constriction, thereby raising the blood pressure. α₁ blockers include prazosin and terazosin. They may also cause a small reduction in blood cholesterol levels.

Beta blockers are widely used in the treatment of high blood pressure (hypertension), certain irregular heart rhythms (arrhythmias), angina pectoris (chest pain associated with insufficient oxygen delivery to the heart), heart attack and heart attack prevention, and heart failure. There are also many non-cardiovascular uses for these drugs. Examples of β blockers include propranolol, metoprolol, and atenolol.

Conformational Model

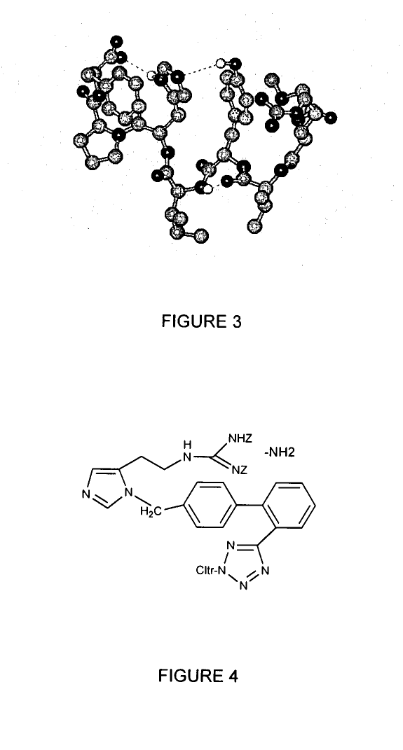

In 1994 a model was developed of Angiotensin II (FIG. 3) which involves an aromatic ring cluster and consequently a charge relay system formed from the triad of aminoacids Tyr⁴-His⁶-Phe⁸. These three aminoacids are a strict requirement for Angiotensin II to exert its agonist activity. Comparative nuclear magnetic resonance studies of the backbone structure between peptide agonists and antagonists have shown that agonists display ring clustering and form a change relay system. Such clustering is also present to the competitive antagonist Tyr(Me)⁴ AngII (Sarmesin) which lacks the potential of the charge relay system and the form of the tyrosinate anion, which is a strict requirement for agonist activity in the proposed model. In addition, the proposed conformation of ANG II overlays the recently discovered non-peptide ANG II receptor antagonist EXP-3174 and its analogs when molecular modeling techniques and superimposition studies are applied. Finally, the ring cluster conformation is supported by the design and synthesis of a novel constrained ANG II cyclic analogue [Sar¹, Lys³, Glu⁵]ANG II, which possesses agonist activity when tested in the rat uterus assay and in anesthetized rabbits. This potent cyclic analog was designed to have a major molecular feature the integrity of the ring cluster. Based on structure activity relationships which demand the presence of Phe, Tyr and His for ANG II to possess biological activity it can be inferred that the ability to form a ring cluster and consequently a charge relay system may be the key stereoelectronic molecular features of ANG II for exerting biological activity.

Theoretical calculations further improved the model and the revised one includes electrostatic interactions between Asp¹-Arg² and Arg2-Tyr⁴.

Rationale for Use of Combined ANGII Blockade and Central Sympathetic Suppression in Chronic Congestive Heart Failure (CHF)

Neurohormonal activation is the hallmark of decompensated chronic CHF. During episodes of decompensation there is stimulation of several pressor hormones, including the renin-angiotensin system, the sympathetic nervous system, vasopressin, endothelin and probably other systemically and locally acting neurohumoral factors. This stimulation is compensatory and is teleologically meant to sustain circulatory homeostasis in the face of falling systolic pressure due to myocardial pump failure. However, the resulting peripheral vasoconstriction increases systemic vascular resistance and further impedes the cardiac function with this additional hemodynamic burden, thus exhausting the already ailing myocardium.

Treatment of CHF by suppression of the renin-angiotensin system was first proposed in the early 1970s and shown to break this vicious circle by lowering systemic vascular resistance and selectively improving the coronary blood flow and the renal and cerebral perfusion. Subsequent multicenter formal trials have now established this approach as the only treatment proven to diminish mortality in CHF. Sympathetic suppression has been attempted by various means over the past several years. In most cases, it was effected by means of selective peripheral α and/or β adrenergic receptor blockade, with generally mixed results. In the past, central sympathetic suppression was thought to be inappropriate for this purpose, for fear of a presumed negative inotropic effect. However, over the past three years we have conducted a number of short-term and long-term studies where we have documented hemodynamic improvement and enhanced electrical stability in CHF patients treated with the central sympathoinhibitory drug clonidine for up to 23 months. These studies have rekindled the interest of the scientific community in this approach. Nevertheless, at this time there are no controlled long-term clinical trials to prove the benefits of this approach, as anticipated on the basis of the hemodynamic and electrophysiologic data.

In one short-term study we evaluated the combined results of the angiotensin converting enzyme (ACE) inhibitor, captopril, and clonidine given alone or in combination. The data suggest that the ACE inhibitor mostly improved the afterload parameters, whereas clonidine mostly improved the preload parameters, and that the combination of the two seems to produce additive effects superior to each agent alone. There is also a recent publication suggesting that ANG II receptor blockade is at least as good an alternative to ACE inhibition and may actually offer advantages over the standard ACE inhibition in CHF. The next logical step is to study the effects of a single agent with combined AT receptor blocking properties plus central sympathoinhibitory properties. Combination of these properties in a single long-acting agent would greatly simplify therapy and improve compliance in such patients, especially those who are treated with polypharmacy due to coexisting conditions requiring multiple dosing schedules. This combination should prove advantageous in the treatment of CHF and other conditions, such as hypertension and diabetic nephropathy.

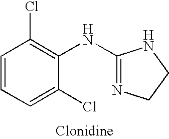

Clinical Perspective

(A) Angiotensin II Receptor Antagonists

Angiotensin Receptor Antagonists (ARA) have effects similar to ACE inhibitors, which are widely used to treat hypertension and congestive heart failure. In the previous decade, two ACE inhibitors cartopril and enalapril, have had featured in the top-five selling drugs worldwide. However, an important issue favoring the future clinical application of angiotensin antagonists is their ability to decrease the incidence of side effects compared to other cardiovascular drugs, including ACE inhibitors. In controlled clinical trials on the first angiotensin antagonist to be introduced, namely losartan (tradename Cozaar), involving over 2500 patients with essential hypertension, the only drug-adverse event with an incidence greater than placebo was dizziness (2.4% of patients on Cozaar versus 1.3% with placebo). Most importantly, the incidence of “dry cough”, a disturbing side effect occurring in up to 20% of the patients taking ACE inhibitors, was absent in patients taking Cozaar. The absence of “dry cough” alone could perhaps provide the ARAs with a sufficient advantage to usurp the ACE inhibitors and make angiotensin inhibitors the highest selling cardiovascular drugs. The potential disadvantage of ARAs is that they elevate plasma and tissues levels of ANG II, which may act on other angiotensin receptors (e.g. AT2) and thus mediate undesirable side effects.

(B) Angiotensin II Receptor Antagonists with Sympathetic Suppressant Properties in Future Hypertension Therapy as Substituents of Individual Treatment

Maintenance of abnormally elevated peripheral vascular resistance in hypertension and Chronic Heart Failure (CHF) is due in part to angiotensin II (RAS) and to the Sympathetic Nervous System (SNS). Over the past 25 years several approaches have been employed to inhibit the formation or the activities (via receptor blockade) of either the Ang II or the SNS in order to treat hypertension and/or CHF. Gavras and his collaborators pioneered these efforts by conducting the first clinical studies demonstrating the hemodynamic benefits of Ang II inhibition. This treatment has now become standard in chronic CHF and is the only treatment shown to diminish morbidity and mortality. Central SNS suppression with clonidine has been used for over 30 years to treat hypertension. In a series of recent studies, we investigated the use of clonidine for the treatment of CHF alone or in combination with Ang II inhibition, the latter achieved by administering the two agents concurrently. These dual action substances, are expected to dominate the market in future hypertension therapies over individual treatments.

The present invention seeks to provide potent, non-peptide hybrid compounds which combine the most important pharmacological characteristics of both the AT1 antagonists and the α₂ adrenergic agonists.

### STATEMENT OF INVENTION

A first aspect of the invention relates to a compound of formula I,

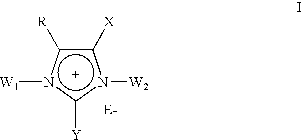

wherein

R is H, halogen;

Y is H, CH₂O-alkyl, CH₂S-alkyl, CH₂OH, CH₂SH, CH₂-halogen, COOH, halogen or CHO;

X is (CH₂)ₙR₁, wherein R₁ is —NH₂, NHR′, —NH—C(═NH)NH₂, NH—C(═NR′)—NHR″ or

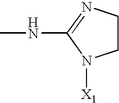

wherein X₁ is —COOCMe₃, —COMe, —COEt, COPh, trityl, halotrityl or benzyl;

n is 1 to 10;

R′ and R″ are each independently alkyl, cycloalkyl, alkyl-cycloalkyl, or an amino or guanadino nitrogen protecting group, PG₁, or R′ and R″ are linked to form a cyclic group;

W₁ and W₂ are each independently —(CH₂)ₘ—K—Z—Z₁, where m is 1 to 5;

K is biphenyl or monophenyl;

Z is tetrazolyl or COO—;

Z₁ is H, trityl, halotrityl, CH₂(Ph), COOH, COO-alkyl or CH(Ph)₂, wherein each Ph group is optionally substituted by one or more halogens; and

E is an anion;

or a pharmaceutically acceptable salt thereof.

Advantageously, the above dialkylated compounds of formula I are lipophilic, thus rendering them particularly suitable for transdermal administration.

A second aspect of the invention relates to a compound of formula IIa or IIb,

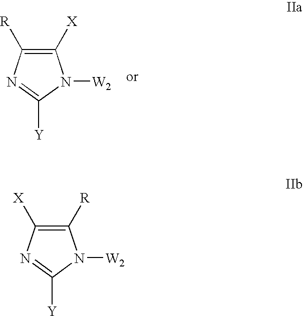

wherein

R is H, halogen;

Y is H, CH₂O-alkyl, CH₂S-alkyl, CH₂OH, CH₂SH, CH₂-halogen, COOH, halogen or CHO;

X is (CH₂)ₙR₁, wherein R₁ is —NH₂, —NH—C(═NH)NH₂, NH—C(═NR′)—NHR″ or

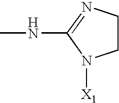

wherein X₁ is —COOCMe₃, —COMe, —COEt, COPh, trityl, halotrityl or benzyl;

n is 1 to 10;

R′ and R″ are each independently alkyl, cycloalkyl, alkyl-cycloalkyl, or an amino or guanadino nitrogen protecting group, PG₁, or R′ and R″ are linked to form a cyclic group;

W₂ is —(CH₂)ₘ—K—Z—Z₁, where m is 1 to 5;

K is biphenyl or monophenyl;

Z is tetrazolyl or COO—; and

Z₁ is H, trityl, halotrityl, CH₂(Ph), COOH, COO-alkyl or CH(Ph)₂, wherein each Ph group is optionally substituted by one or more halogens;

or a pharmaceutically acceptable salt thereof.

A third aspect of the invention relates to a pharmaceutical composition comprising a compound as defined above, or a pharmaceutically acceptable salt thereof, admixed with a pharmaceutically acceptable diluent, excipient or carrier.

A fourth aspect of the invention relates to a process for preparing compounds as defined above.

A fifth aspect of the invention relates to the use of a compound as described above, or a pharmaceutically acceptable salt thereof, in the preparation of a medicament for treating hypertension or a cardiovascular disorder.

A sixth aspect of the invention relates to a method of treating hypertension or a cardiovascular disorder in a subject, said method comprising administering to the to subject a therapeutically effective amount of a compound as described above, or a pharmaceutically acceptable salt thereof.

A seventh aspect of the invention relates to a compound as defined above for use in medicine.

An eighth aspect of the invention relates to a compound as defined above for treating hypertension or a cardiovascular disorder.

In particular, the invention relates to:

(A) derivatives of 4(5)-substituted imidazole (histamine) of the chemical structure described herein which act as angiotensin antagonists useful in the treatment of certain cardiovascular diseases;

(B) a synthetic route which provides potent structures in a short pathway, starting from histamine and bromomethyl biphenyl (or phenyl) trityl tetrazole (or 2-bromomethyl biphenyl tetrazole derivatives) as the alkylating reagent; and

(C) the utility of these antagonists in hydrophilic or lipophilic form for treatment of hypertension through simultaneous suppression of the RAS (Renin Angiotensin II System) and the SS (Sympathetic System) systems.

Preferred synthesized compounds have been given the names H—P, E-P and H-Px and are capable of selectively blocking angiotensin II and at the same time acting as α₂ adrenergic agonists. These novel compounds are proprietary new drugs, which have potential applications in the treatment of hypertension, congestive heart failure, diabetic nephropathy and other cardiovascular diseases.

Advantageously, the final products from starting materials are achieved in a six to seven step high yielding synthesis. A novel cost effective synthetic strategy has been developed in our laboratory allowing facile synthesis of 1,5-disubstituted imidazoles with dual activity. The compounds so produced are “hybrid drugs” which are well absorbed in the gut with very high antihypertensive potency in both RAS and SS.

Rationale for Use of Histamine

In the present invention we use histamine instead of 4(5)-butylimidazole which is the basis for the synthesis of Losartan analogues.

In histamine based analogues, the butyl and hydroxymethyl groups of losartan are reversed, while the butyl group is replaced by alkyl-amino group or alkyl-guanidino group. Introducing a basic group enhances the affinity of the analogue for its receptor and increases the inhibitory effect compared to analogues with butyl group. In view of the basic group, these analogues exhibit clonidine like activity showing sympathetic suppressant properties. Furthermore, using histamine as a starting material allows the cost effective synthesis of potent AT1 antagonists through 1,5-disubstituted imidazoles in four high yielding steps.

Structure-Activity Studies

Reorientation of the imidazole ring of losartan provided novel compounds that treat hypertension in anesthetized rats and rabbits.

Based on a survey of four of the possible five orientations of the imidazole ring of losartan, it was observed that compounds in which the biphenyl moiety is attached to an imidazole N atom, rather than one of the C atoms of the heterocyclic ring, have the highest activity. With this knowledge at hand we developed a series of compounds in an attempt to attain the high biological activities observed for losartan itself. Transposing the substituents at the 2 and 5 positions of the imidazole ring of losartan has provided compounds with significant activities in vitro when examined in the rat isolated uterus assay. Further protection of tetrazole by protecting groups as Trt, Cl-Trt, benzyl and derivatives thereof, further increased the activity in this assay, as well as in anesthetized rats and rabbits.

Furthermore, replacement of the alkyl group with alkylamino or alkyl guanidino groups have provided substances which retain high antagonist activity for angiotensin II induced hypertension, whilst simultaneously suppressing the SNS.

Such observations clearly illustrate that the orientation of the imidazole ring contributes to the activity of these Ang II receptor antagonists in a manner that is, at present, unclear. One possible explanation is that the biphenyl ring system is acting as a template, and the tetrazole and imidazole rings are the pharmacophores. If this were the case, then rotation of the imidazole ring would effect biological activity. Synthesis of potent substance 9 (FIG. 4) is characterized by low yields and formation of stereoisomers, thereby rendering it unsuitable for large scale production.

### DETAILED DESCRIPTION

As mentioned above, a first aspect of the invention relates to compounds of formula I, IIa or IIb as defined above which have therapeutic applications as angiotensin II receptor antagonists.

One aspect of the invention relates to a compound of formula I′ or II′,

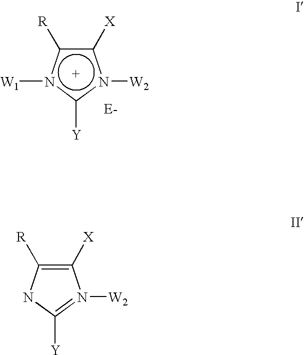

wherein

R is H, halogen;

Y is H, CH₂O-alkyl, CH₂S-alkyl, CH₂OH, CH₂SH or CHO;

X is (CH₂)ₙR₁, wherein R₁ is —NH₂, NHR′, —NH—C(═NH)NH₂, NH—C(═NR′)—NHR″;

n is 1 to 10;

R′ and R″ are each independently alkyl, cycloalkyl, alkyl-cycloalkyl, or an amino or guanadino nitrogen protecting group, PG₁, or R′ and R″ are linked to form a cyclic group;

W₁ and W₂ are each independently —(CH₂)ₘ—K—Z—Z₁, where m is 1 to 5;

K is biphenyl or monophenyl;

Z is tetrazolyl or COO—;

Z₁ is H, trityl, halotrityl, halobenzyl or CH(Ph)₂; and

E is an anion;

or a pharmaceutically acceptable salt thereof.

For compounds of formula I, Ia, Ib, I′ and II″, the following definitions apply.

As used herein, the term “alkyl” includes both saturated straight chain and branched alkyl groups. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

As used herein, the term “cycloalkyl” refers to a cyclic alkyl group. Preferably, the cycloalkyl group is a C₃₋₁₂ cycloalkyl group.

As used herein, the term “aryl” refers to a substituted (mono- or poly-) or unsubstituted monoaromatic or polyaromatic system, wherein said polyaromatic system may be fused or unfused. Preferably, the term “aryl” is includes groups having from 6 to 10 carbon atoms, e.g. phenyl, naphthyl etc. The term “aryl” is synonymous with the term “aromatic”.

The term “aralkyl” is used as a conjunction of the terms alkyl and aryl as given above. Preferred aralkyl groups include CH₂Ph and CH₂CH₂Ph and the like.

As used herein, protecting groups PG₁ or PG refer to any suitable protecting group for amino or guanidino nitrogens. Such protecting groups will be familiar to the skilled artisan and preferred groups include Fmoc, Boc and COCF₃. Further details of suitable N-protecting groups may be found in “Protective Groups in Organic Synthesis” by Peter G. M. Wuts and Theodoro W. Greene, 2^{nd} Edition).

In one preferred embodiment of the invention, X is (CH₂)ₙ—NH₂, (CH₂)—NH—C(═NH)NH₂ or (CH₂)ₙ—R₁, where R₁ is

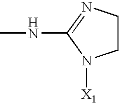

and wherein X₁ is —COOCMe₃, —COMe, —COEt, COPh, trityl, halotrityl or benzyl.

In one preferred embodiment of the invention, X is (CH₂)ₙ—R₁, wherein R₁ is

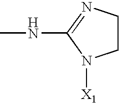

and wherein X₁ is —COOCMe₃, —COMe, —COEt, COPh, trityl, halotrityl or benzyl.

In another preferred embodiment of the invention, X is (CH₂)ₙ—NH₂.

In another preferred embodiment of the invention, X is (CH₂)ₙ—NH(C═NH)NH₂.

In one preferred embodiment, n is 1 to 5. More preferably n is 1 or 2.

In one highly preferred embodiment, n is 2.

In one preferred embodiment, the compound is of formula I.

In one preferred embodiment, the anion E⁻is a halo ion, more preferably Br⁻.

In one preferred embodiment, W₁═W₂.

In one particularly preferred embodiment, W₁ is

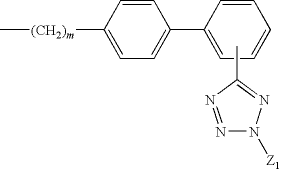

In another particularly preferred embodiment, W₁ is

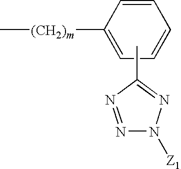

Preferably, m is 1.

In one preferred embodiment, Y is H, CH₂OH, CH₂OMe, CH₂OEt, CH₂SH, CH₂SMe, halogen or CH₂SEt.

Even more preferably, Y is CH₂OH or H.

In one preferred embodiment, R is H, Cl, Br, F or I. More preferably, R is H or Cl, more preferably H.

In one preferred embodiment, Z₁ is H, trityl, halotrityl, dibenzyl or benzyl.

More preferably, Z₁ is H, trityl, chlorotrityl or benzyl, even more preferably H or trityl.

In one highly preferred embodiment, the compound is of formula E,

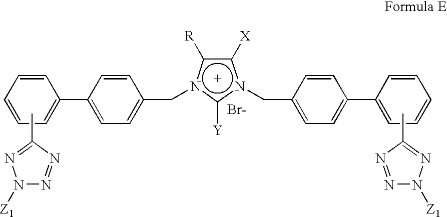

wherein:

X is (CH₂)ₙ—NH—C(═NH)NH₂, (CH₂)ₙ—NH₂ or (CH₂)ₙ—R₁ where R₁ is

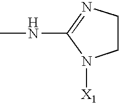

wherein X₁ is —COOCMe₃, —COMe, —COEt, COPh, trityl, halotrityl or benzyl;

n is 1 to 5;

Y is H, CH₂O-alkyl, CH₂S-alkyl, CH₂OH, CH₂SH or CHO;

R is H or halogen; and

Z₁ is H, trityl, 2-chlorotrityl or benzyl.

In another highly preferred embodiment, the compound is of formula F,

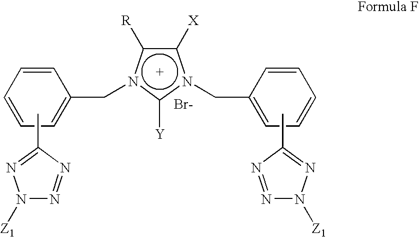

wherein:

X is (CH₂)ₙ—NH—C(═NH)NH₂, (CH₂)ₙ—NH₂ or (CH₂)ₙ—R₁ where R₁ is

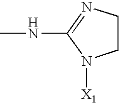

wherein X₁ is —COOCMe₃, —COMe, —COEt, COPh, trityl, halotrityl or benzyl;

n is 1 to 5;

Y is H, CH₂O-alkyl, CH₂S-alkyl, CH₂OH, CH₂SH or CHO;

R is H or halogen; and

Z₁ is H, trityl, 2-chlorotrityl or benzyl.

Preferably, for compounds of formula I, IIa and IIb, R is H.

Preferably, for compounds of formula I, IIa and IIb, Y is H or CH₂OH.

In one preferred embodiment, the compound of formula I, IIa and IIb is in the form of a pharmaceutically acceptable salt. In one highly preferred embodiment, the compound is in the form of the trifluoroacetic acid salt.

In one preferred embodiment, the compound is of formula IIa or IIb.

In one highly preferred embodiment, the compound is of formula IIa.

In another embodiment, the invention relates to a class of novel 1-biphenyl-5-imidazole derivatives, represented by formula A,

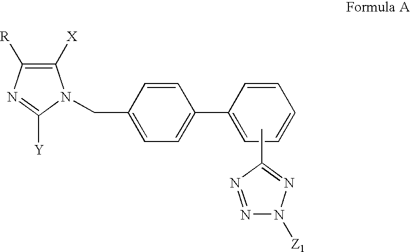

wherein

Y is H, CH₂O-alkyl, CH₂S-alkyl, CH₂OH, CH₂SH or CHO;

R═H, halogen;

X is (CH₂)ₙ—NH₂;

n is 1 to 5; and

Z₁ is H, trityl, 2-chlorotrityl or benzyl.

In another embodiment, this invention relates to a class of novel 1-monophenyl-5-imidazole derivatives as represented by formula B,

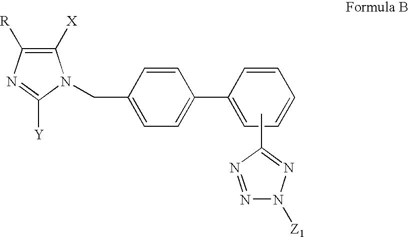

wherein

Y is H, CH₂O-alkyl, CH₂S-alkyl, CH₂OH, CH₂SH or CHO;

R═H, halogen;

X is (CH₂)ₙ—NH—C(═NH)NH₂;

n is 1 to 5; and

Z₁ is H, trityl, 2-chlorotrityl or benzyl.

In another embodiment, this invention relates to a class of novel 1-monophenyl-5-imidazole derivatives as represented by formula C.

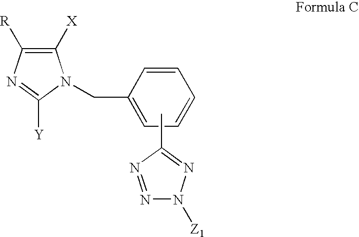

wherein

Y is H, CH₂O-alkyl, CH₂S-alkyl, CH₂OH, CH₂SH or CHO;

R═H, halogen;

X is (CH₂)ₙ—NH₂;

n is 1 to 5; and

Z₁ is H, trityl, 2-chlorotrityl or benzyl.

In another embodiment, this invention relates to a class of novel 1-monophenyl-5-imidazole derivatives as represented by formula D.

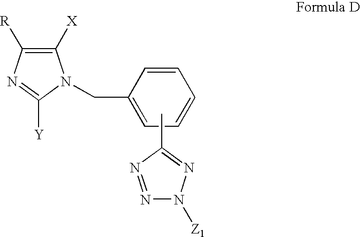

wherein

Y is H, CH₂O-alkyl, CH₂S-alkyl, CH₂OH, CH₂SH or CHO;

R═H, halogen;

X is (CH₂)ₙ—NH—C(═NH)NH₂;

n is 1 to 5; and

Z₁ is H, trityl, 2-chlorotrityl or benzyl.

In one preferred embodiment, the compound of the invention is represented by formula G:

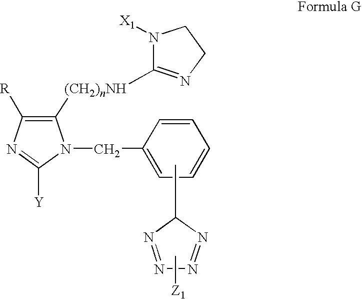

wherein

Y is H, CH₂O-alkyl, CH₂S-alkyl, CH₂OH, CH₂SH or CHO;

R═H, halogen;

X₁ is —COOCMe₃, —COMe, —COEt, COPh, trityl, halotrityl or benzyl;

n is 1 to 5; and

Z₁ is H, trityl, 2-chlorotrityl or benzyl.

In one preferred embodiment, the compound of the invention is represented by formula H:

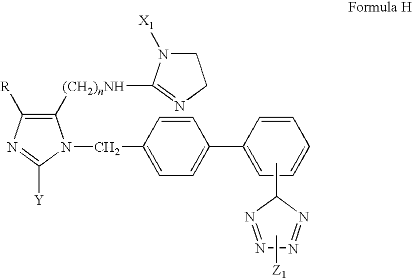

wherein

Y is H, CH₂O-alkyl, CH₂S-alkyl, CH₂OH, CH₂SH or CHO;

R═H, halogen;

X₁ is —COOCMe₃, —COMe, —COEt, COPh, trityl, halotrityl or benzyl;

n is 1 to 5; and

Z₁ is H, trityl, 2-chlorotrityl or benzyl.

In one highly preferred embodiment, the compound of the invention is selected from:

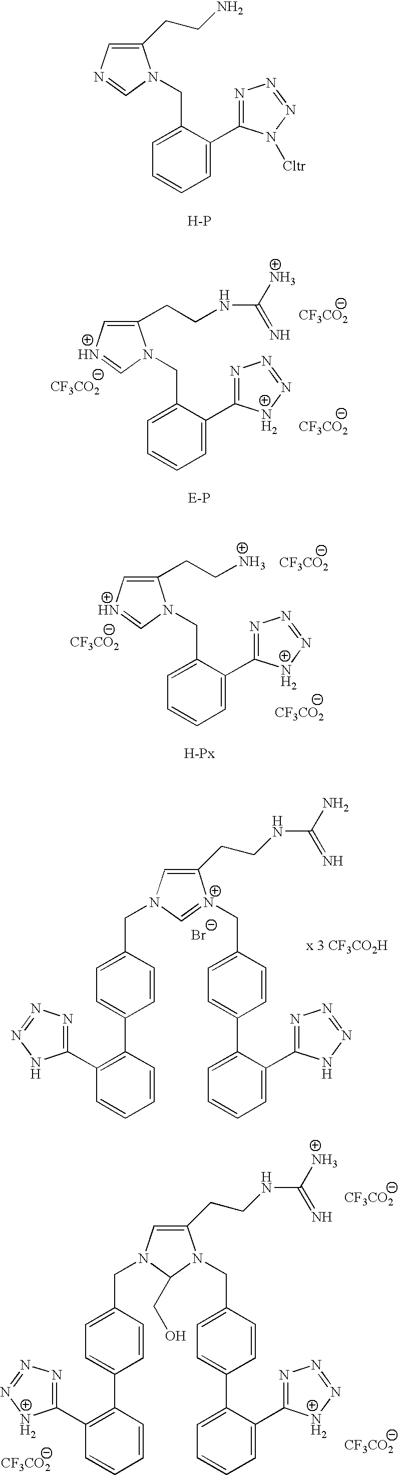

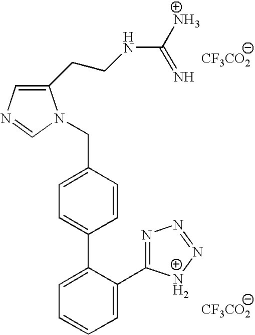

In an even more highly preferred embodiment, the compound of the invention is selected from: H—P, E-P and H-Px.

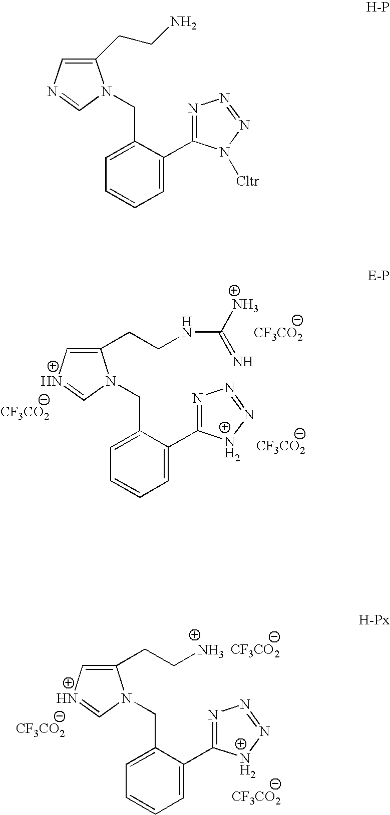

In one highly preferred embodiment of the invention, the compound is of formula A′, B′, C′, D′, E′, F′, G′ or H′ as set forth below.

In one preferred embodiment, the compound of the invention is of formula A′:

Formula A′

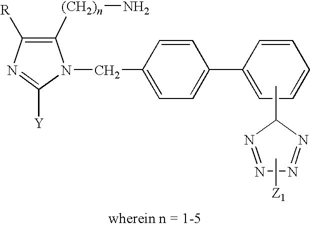

| Compound No. | Y | R | Z₁ |
|---|---|---|---|
| 1 | H | H | H |
| 2 | H | H | Trt |
| 3 | H | Cl | H |
| 4 | H | Cl | Trt |
| 5 | H O CH₂ | H | H |
| 6 | H O CH₂ | H | Trt |
| 7 | H O CH₂ | Cl | H |
| 8 | H O CH₂ | Cl | Trt |
| 9 | Me O CH₂ | H | H |
| 10 | Me O CH₂ | H | Trt |
| 11 | Me O CH₂ | Cl | H |
| 12 | Me O CH₂ | Cl | Trt |
| 13 | Et O CH₂ | H | H |
| 14 | Et O CH₂ | H | Trt |
| 15 | Et O CH₂ | Cl | H |
| 16 | Et O CH₂ | Cl | Trt |
| 17 | H S CH₂ | H | H |
| 18 | H S CH₂ | H | Trt |
| 19 | Me S CH₂ | Cl | H |
| 20 | Me S CH₂ | Cl | Trt |
| 21 | Et S CH₂ | Cl | H |
| 22 | Et S CH₂ | Cl | Trt |

In one preferred embodiment, the compound of the invention is of formula B′:

Formula B′

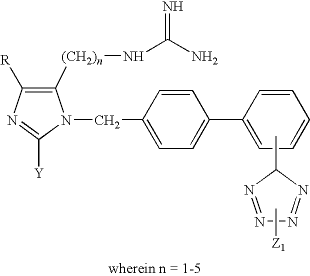

| Compound No. | Y | R | Z₁ |
|---|---|---|---|
| 23 | H | H | H |
| 24 | H | H | Trt |
| 25 | H | Cl | H |
| 26 | H | Cl | Trt |
| 27 | H O CH₂ | H | H |
| 28 | H O CH₂ | H | Trt |
| 29 | H O CH₂ | Cl | H |
| 30 | H O CH₂ | Cl | Trt |
| 31 | Me O CH₂ | H | H |
| 32 | Me O CH₂ | H | Trt |
| 33 | Me O CH₂ | Cl | H |
| 34 | Me O CH₂ | Cl | Trt |
| 35 | Et O CH₂ | H | H |
| 36 | Et O CH₂ | H | Trt |
| 37 | Et O CH₂ | Cl | H |
| 38 | Et O CH₂ | Cl | Trt |
| 39 | H S CH₂ | H | H |
| 40 | H S CH₂ | H | Trt |
| 41 | Me S CH₂ | Cl | H |
| 42 | Me S CH₂ | Cl | Trt |
| 43 | Et S CH₂ | Cl | H |
| 44 | Et S CH₂ | Cl | Trt |

In one preferred embodiment, the compound of the invention is of formula C′:

Formula C′

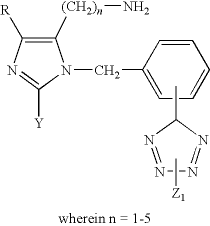

| Compound No. | Y | R | Z₁ |
|---|---|---|---|
| 45 | H | H | H |
| 46 | H | H | Trt |
| 47 | H | Cl | H |
| 48 | H | Cl | Trt |
| 49 | H O CH₂ | H | H |
| 50 | H O CH₂ | H | Trt |
| 51 | H O CH₂ | Cl | H |
| 52 | H O CH₂ | Cl | Trt |
| 53 | Me O CH₂ | H | H |
| 54 | Me O CH₂ | H | Trt |
| 55 | Me O CH₂ | Cl | H |
| 56 | Me O CH₂ | Cl | Trt |
| 57 | Et O CH₂ | H | H |
| 58 | Et O CH₂ | H | Trt |
| 59 | Et O CH₂ | Cl | H |
| 60 | Et O CH₂ | Cl | Trt |
| 61 | H S CH₂ | H | H |
| 62 | H S CH₂ | H | Trt |
| 63 | Me S CH₂ | Cl | H |
| 64 | Me S CH₂ | Cl | Trt |
| 65 | Et S CH₂ | Cl | H |
| 66 | Et S CH₂ | Cl | Trt |

In one preferred embodiment, the compound of the invention is of formula D′:

Formula D′

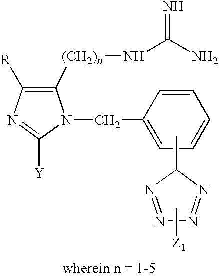

| Compound No. | Y | R | Z₁ |
|---|---|---|---|
| 67 | H | H | H |
| 68 | H | H | Trt |
| 69 | H | Cl | H |
| 70 | H | Cl | Trt |
| 71 | H O CH₂ | H | H |
| 72 | H O CH₂ | H | Trt |
| 73 | H O CH₂ | Cl | H |
| 74 | H O CH₂ | Cl | Trt |
| 75 | Me O CH₂ | H | H |
| 76 | Me O CH₂ | H | Trt |
| 77 | Me O CH₂ | Cl | H |
| 78 | Me O CH₂ | Cl | Trt |
| 79 | Et O CH₂ | H | H |
| 80 | Et O CH₂ | H | Trt |
| 81 | Et O CH₂ | Cl | H |
| 82 | Et O CH₂ | Cl | Trt |
| 83 | H S CH₂ | H | H |
| 84 | H S CH₂ | H | Trt |
| 85 | Me S CH₂ | Cl | H |
| 86 | Me S CH₂ | Cl | Trt |
| 87 | Et S CH₂ | Cl | H |
| 88 | Et S CH₂ | Cl | Trt |

In one preferred embodiment, the compound of the invention is of formula E′:

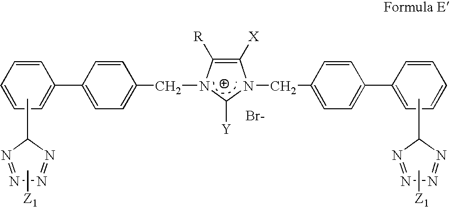

wherein:
X═—(CH₂)ₙ—NH—C(═NH)NH₂;
   —(CH₂)ₙ—NH₂; or
   —(CH₂)ₙ—R₁, where R₁ is

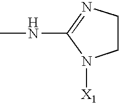

and X₁ is —COOCMe₃, —COMe, —COEt, COPh, trityl, halotrityl or benzyl;
n=1-5

| Compound No | Y | R | Z₁ |
|---|---|---|---|
| 89 | H | H | H |
| 90 | H | H | Trt |
| 91 | H | Cl | H |
| 92 | H | Cl | Trt |
| 93 | HOCH₂ | H | H |
| 94 | HOCH₂ | H | Trt |
| 95 | HOCH₂ | Cl | H |
| 96 | HOCH₂ | Cl | Trt |
| 97 | MeOCH₂ | H | H |
| 98 | MeOCH₂ | H | Trt |
| 99 | MeOCH₂ | Cl | H |
| 100 | MeOCH₂ | Cl | Trt |
| 101 | EtOCH₂ | H | H |
| 102 | EtOCH₂ | H | Trt |
| 103 | EtOCH₂ | Cl | H |
| 104 | EtOCH₂ | Cl | Trt |
| 105 | HSCH₂ | H | H |
| 106 | HSCH₂ | H | Trt |
| 107 | MeSCH₂ | Cl | H |
| 108 | MeSCH₂ | Cl | Trt |
| 109 | EtSCH₂ | Cl | H |
| 110 | EtSCH₂ | Cl | Trt |

In one preferred embodiment, the compound of the invention is of formula F′:

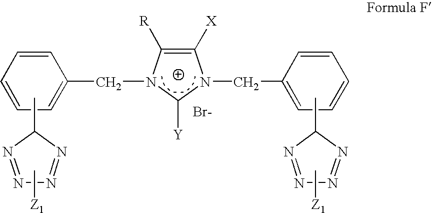

wherein:
X═—(CH₂)ₙ—NH—C(═NH)NH₂;
   —(CH₂)ₙ—NH₂; or
   —(CH₂)ₙ—R₁, where R₁ is

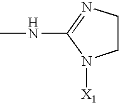

and X₁ is —COOCMe₃, —COMe, —COEt, COPh, trityl, halotrityl or benzyl;
n=1-5;

| Compound No | Y | R | Z₁ |
|---|---|---|---|
| 111 | H | H | H |
| 112 | H | H | Trt |
| 113 | H | Cl | H |
| 114 | H | Cl | Trt |
| 115 | HOCH₂ | H | H |
| 116 | HOCH₂ | H | Trt |
| 117 | HOCH₂ | Cl | H |
| 118 | HOCH₂ | Cl | Trt |
| 119 | MeOCH₂ | H | H |
| 120 | MeOCH₂ | H | Trt |
| 121 | MeOCH₂ | Cl | H |
| 122 | MeOCH₂ | Cl | Trt |
| 123 | EtOCH₂ | H | H |
| 124 | EtOCH₂ | H | Trt |
| 125 | EtOCH₂ | Cl | H |
| 126 | EtOCH₂ | Cl | Trt |
| 127 | HSCH₂ | H | H |
| 128 | HSCH₂ | H | Trt |
| 129 | MeSCH₂ | Cl | H |
| 130 | MeSCH₂ | Cl | Trt |
| 131 | EtSCH₂ | Cl | H |
| 132 | EtSCH₂ | Cl | Trt |

In one preferred embodiment, the compound of the invention is of formula G′:

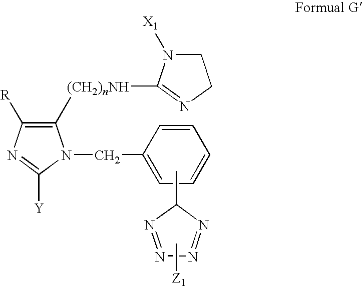

wherein n=1-5;
X₁ is —COOCMe₃, —COMe, —COEt, COPh, trityl, halotrityl or benzyl

| Compound No. | Y | R | Z₁ |
|---|---|---|---|
| 133 | H | H | H |
| 134 | H | H | Trt |
| 135 | H | Cl | H |
| 136 | H | Cl | Trt |
| 137 | HOCH₂ | H | H |
| 138 | HOCH₂ | H | Trt |
| 139 | HOCH₂ | Cl | H |
| 140 | HOCH₂ | Cl | Trt |
| 141 | MeOCH₂ | H | H |
| 142 | MeOCH₂ | H | Trt |
| 143 | MeOCH₂ | Cl | H |
| 144 | MeOCH₂ | Cl | Trt |
| 145 | EtOCH₂ | H | H |
| 146 | EtOCH₂ | H | Trt |
| 147 | EtOCH₂ | Cl | H |
| 148 | EtOCH₂ | Cl | Trt |
| 149 | HSCH₂ | H | H |
| 150 | HSCH₂ | H | Trt |
| 151 | MeSCH₂ | Cl | H |
| 152 | MeSCH₂ | Cl | Trt |
| 153 | EtSCH₂ | Cl | H |
| 154 | EtSCH₂ | Cl | Trt |

In one preferred embodiment, the compound of the invention is of formula H′:

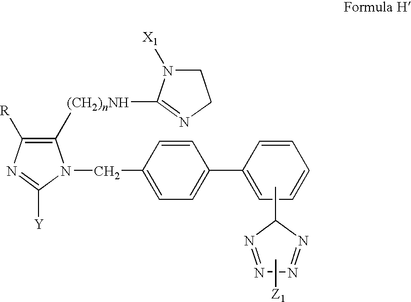

wherein n=1-5;
X₁ is —COOCMe₃, —COMe, —COEt, COPh, trityl, halotrityl or benzyl

| Compound No. | Y | R | Z₁ |
|---|---|---|---|
| 155 | H | H | H |
| 156 | H | H | Trt |
| 157 | H | Cl | H |
| 158 | H | Cl | Trt |
| 159 | HOCH₂ | H | H |
| 160 | HOCH₂ | H | Trt |
| 161 | HOCH₂ | Cl | H |
| 162 | HOCH₂ | Cl | Trt |
| 163 | MeOCH₂ | H | H |
| 164 | MeOCH₂ | H | Trt |
| 165 | MeOCH₂ | Cl | H |
| 166 | MeOCH₂ | Cl | Trt |
| 167 | EtOCH₂ | H | H |
| 168 | EtOCH₂ | H | Trt |
| 169 | EtOCH₂ | Cl | H |
| 170 | EtOCH₂ | Cl | Trt |
| 171 | HSCH₂ | H | H |
| 172 | HSCH₂ | H | Trt |
| 173 | MeSCH₂ | Cl | H |
| 174 | MeSCH₂ | Cl | Trt |
| 175 | EtSCH₂ | Cl | H |
| 176 | EtSCH₂ | Cl | Trt |

Therapeutic Use

The compounds of the present invention have been found to inhibit angiotensin II activity and are therefore believed to be of use in the treatment of hypertension and other cardiac disorders.

As used herein the phrase “preparation of a medicament” includes the use of a compound of the invention directly as the medicament in addition to its use in a screening programmed for further therapeutic agents or in any stage of the manufacture of such a medicament.

In one preferred embodiment, the cardiovascular disorder is chronic congestive heart failure.

In one preferred embodiment, the medicament is in a form suitable for topical or transdermal administration. More preferably, the medicament is in the form of a transdermal patch.

In another preferred embodiment, the medicament is in a form suitable for oral administration.

Another aspect of the invention relates to a method of treating hypertension or a cardiovascular disorder in a subject, said method comprising administering to the subject a therapeutically effective amount of a compound of formula I or II as defined above, or a pharmaceutically acceptable salt thereof.

Preferably, the compound is administered transdermally, more preferably by means of a transdermal patch.

Preferably, the subject is a human.

Another aspect of the invention relates to a compound of formula I or II as defined above for use in medicine.

In another embodiment, this invention concerns a method of treating hypertension in a rabbit anesthetized animal model orally administrating a compound of this invention.

In another embodiment, this invention concerns a method of treating hypertension through transdermal administration.

Pharmaceutical Compositions

Another aspect of the invention relates to a pharmaceutical composition comprising a compound of the invention admixed with a pharmaceutically acceptable diluent, excipient or carrier, or a mixture thereof. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Preferably, the composition is in a form suitable for transdermal administration.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of the present invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of the compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centers and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes solvate forms of the compounds of the present invention. The terms used in the claims encompass these forms.

Polymorphs

The invention furthermore relates to compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

Synthesis

The present invention also relates to a process for preparing compounds of formula I, IIa or IIb as defined above.

Advantageously, the synthesis of the key 1,5-disubstituted imidazole intermediate is achieved in a three step sequence which involves regioselective, clean, high yielding (>85%) reactions.

More specifically, one aspect of the present invention relates to a six step (tritylation of N-1 imidazole and amino group/selective deprotection of amino trityl group/protection by Fmoc/alkylation/removal of trityl groups/removal of Fmoc group) sequence which provides a regioselective, high yielding synthesis of 1,5-disubstituted imidazoles as potential drugs. Suitable protection of tetrazole with Trt, Cl-Trt, Benzyl and derivatives, provides for prodrug substances suitable for treating hypertension and cardiovascular diseases with high activity over an extended duration.

One aspect of the invention relates to a process for preparing a compound of formula IIa as defined above, wherein X is (CH₂)ₙ—NH₂, said process comprising the steps of:

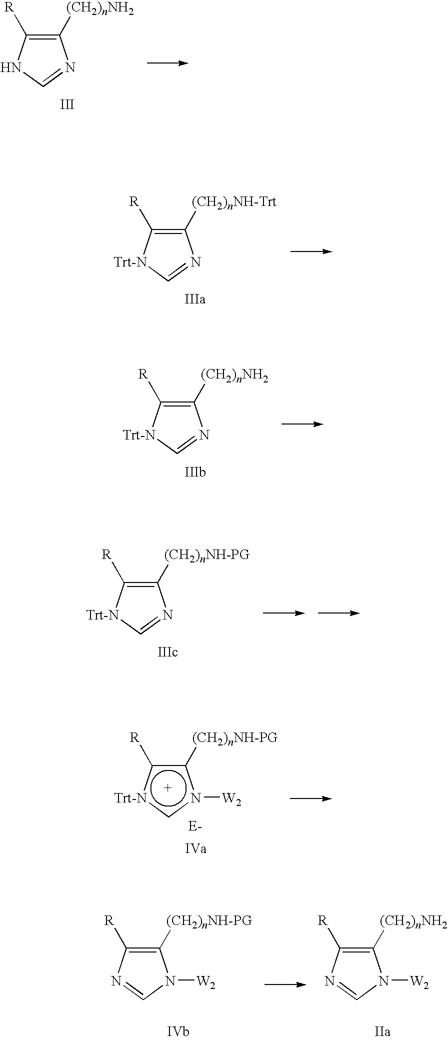

(i) reacting a compound of formula III with trityl chloride to form a compound of formula IIIc;
(ii) reacting said compound of formula IIIa with TFA to form a compound of formula IIIb;
(iii) protecting the free NH₂ group of said compound of formula IIIb with a protecting group, PG, to form a compound of formula IIIc;
(iv) reacting said compound of formula IIIc with Br—(CH₂)ₙ—K—Z—Z₁ to form a compound of formula IVa;
(v) converting said compound of formula IVa to a compound of formula IVb; and
(vi) removing protecting group PG to form a compound of formula IIa.

In one preferred embodiment, step (iv) comprises reacting said compound of formula IIIc with Br—(CH₂)ₙ—K—Z′—Z′₁, wherein Z′ is tetrazoyl and Z′₁ is trityl, chlorotrityl, benzyl or CH(Ph)₂, to form a compound of formula IVa.

In one highly preferred embodiment, Z′₁ is trityl.

In another highly preferred embodiment, Z′₁ is benzyl.

Another aspect of the invention relates to a process for preparing a compound of formula IIa as defined above, wherein X is (CH₂)ₙ—NH—C(═NH)NH₂, said process comprising the steps of:

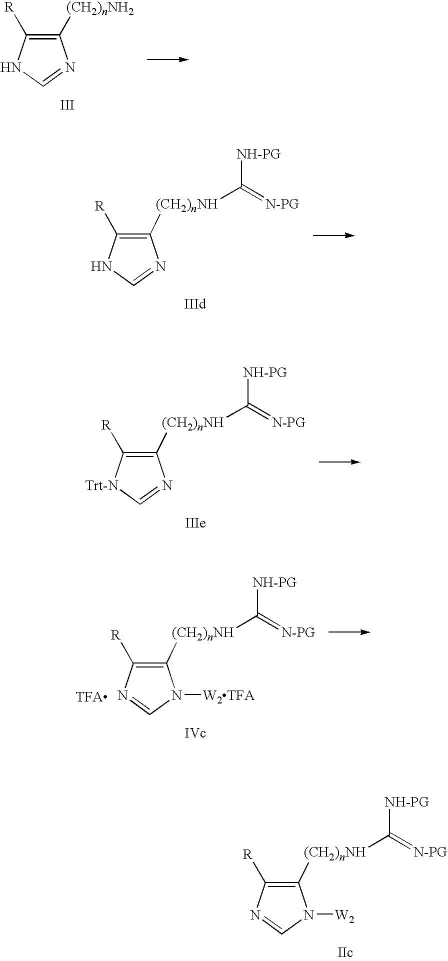

(i) reacting a compound of formula III with PG-HN—C(SMe)=N—PG to form a compound of formula IIId;
(ii) reacting said compound of formula IIId with trityl chloride to form a compound of formula IIIe;
(iii) reacting said compound of formula IIIe with Br—(CH₂)ₙ—K—Z—Z₁ to form a compound of formula IVc;
(iv) converting said compound of formula IVc to a compound of formula IIc; and
(vi) converting said compound of formula IIc to a compound of formula IIa.

In one highly preferred embodiment of the invention, the process is as set forth in

Scheme 1 below:

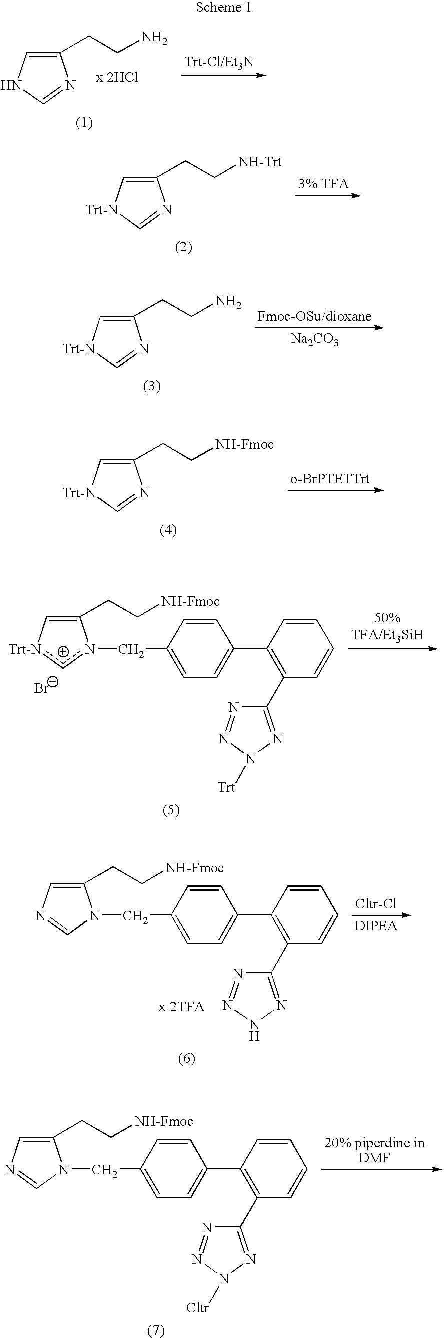

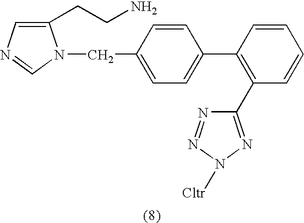

In another highly preferred embodiment of the invention, the process is as set forth in Scheme 2 below:

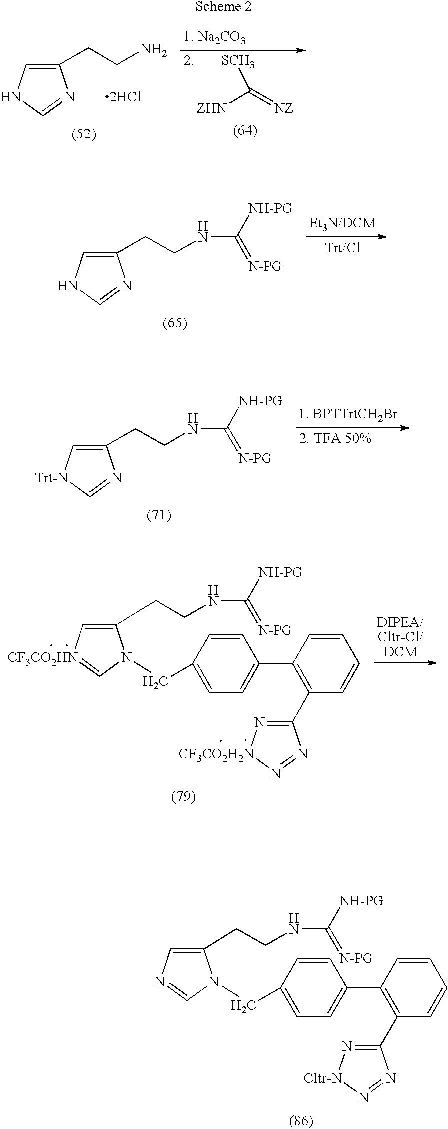

A further aspect of the invention relates to a process for preparing a compound of formula I as defined above, wherein X is (CH₂)ₙ—NH₂, said process comprising the steps of:

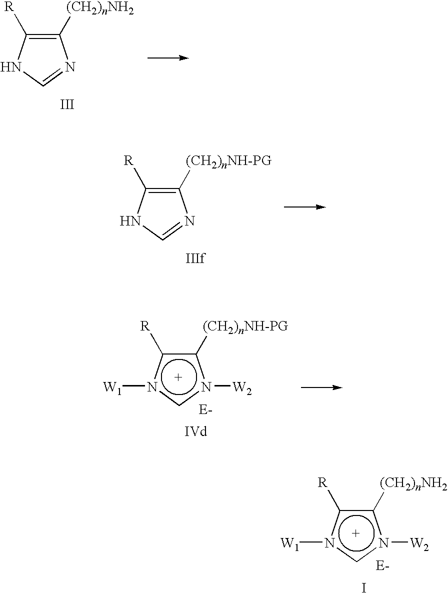

(i) protecting the free NH₂ group of a compound of formula III with a protecting group, PG, to form a compound of formula IIIf;
(ii) reacting said compound of formula IIIf with Br—(CH₂)—K—Z—Z₁ to form a compound of formula IVd; and
(iii) removing protecting group PG from said compound of formula IVd to form a compound of formula I.

Preferably, step (ii) comprises reacting said compound of formula IIIf with Br—(CH₂)ₙ—K—E—Z′₁, wherein Z′ is tetrazoyl and Z′₁ is trityl, chlorotrityl, benzyl or CH(Ph)₂, to form a compound of formula IVc.

More preferably, Z′₁ is trityl or benzyl.

Another aspect of the invention relates to a process for preparing a compound of formula I as defined above, wherein X is (CH₂)ₙ—NH—C(═NH)NH₂, said process comprising the steps of:

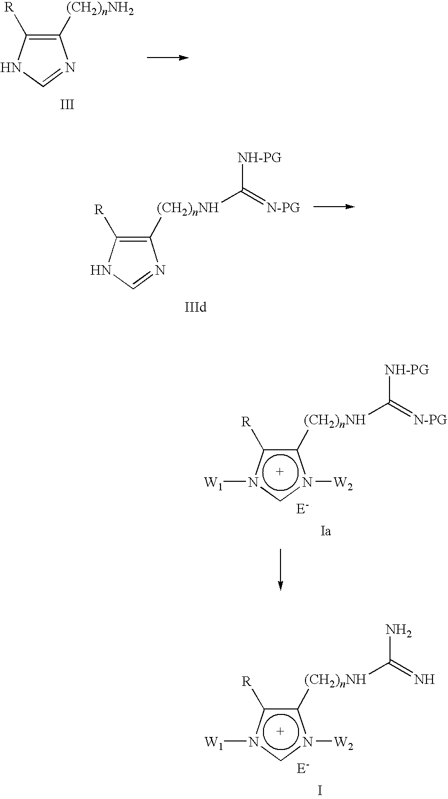

(i) reacting a compound of formula III with PG-HN—C(SMe)=N—PG to form a compound of formula IIId;
(ii) reacting said compound of formula IIId with Br—(CH₂)ₙ—K—Z—Z₁ to form a compound of formula Ia;
(iii) removing protecting groups PG from said compound of formula Ia to form a compound of formula I.

The present invention is further described by way of non-limiting example, and with reference to the following figures, wherein:

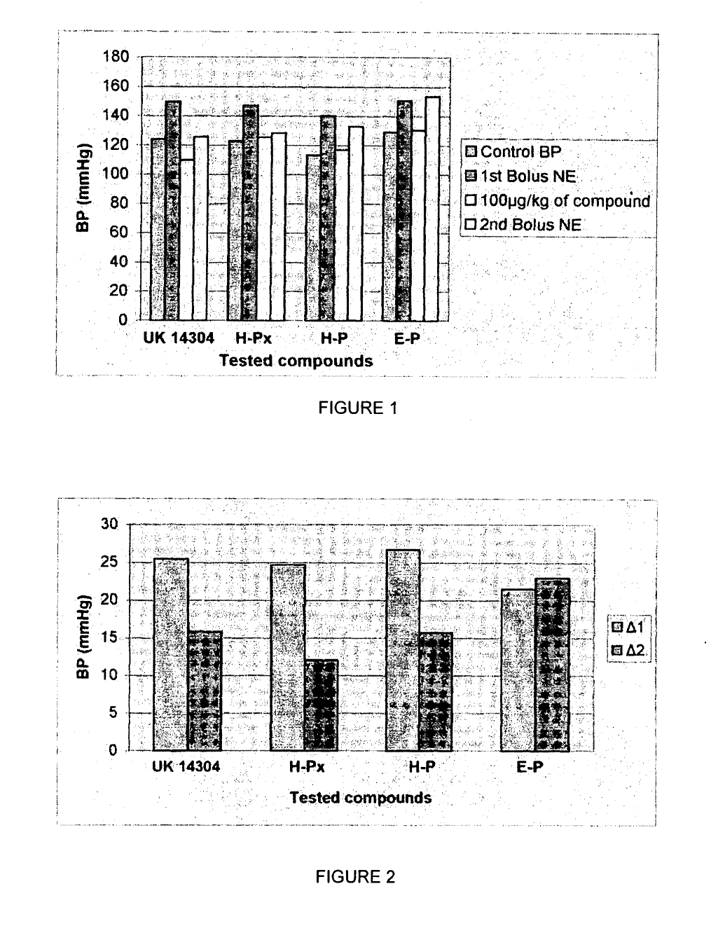

FIG. 1 shows the blood pressure changes during NE and compounds injection.

FIG. 2 shows differences in blood pressure after the first and second injection of NE. (Δ1=BP changes between the first bolus of NE and control BP; Δ2=BP changes between second bolus of NE and the new baseline BP after injection of compound).

FIG. 3 shows a conformational model of Angiotensin II.

FIG. 4 shows potent substance 9 (Elhisartan).

### EXAMPLES

### Novel Synthesis of Elhisartan in Six Steps—Key Steps Synthesis of 3-trityl-4(5) alkyl-amino Derivative of Histamine

### 1.Tritylation of N-3 Imidazole and Amino Group of Histamine

Tritylation of histamine is carried out with trityl chloride in the presence of base (triethylamine) in dichloromethane solution at room temperature (24 h).

### 2. Selective Deprotection of Amino Trityl-Group

Selective deprotection of amino trityl-group is carried out with 3% TFA in DCM solution.

### 3. Protection of Amino Group of Histamine with Fmoc

Protection of amino group of histamine with Fmoc is carried out with Fmoc-OSu in the presence of sodium carbonate in dioxane solution at room temperature (2 h).

### 4. Synthesis of N-Tetrazolylbiphenyl Substituent

The requisite benzyl halide can be prepared by two methods. Treatment of nitrile with trimethyltin azide yields the stannyl tetrazole derivative. This is routinely converted to the trityl derivative, which is brominated with N-bromosuccinimide yielding the corresponding halide.

Alternatively, p-toluyl chloride is converted to the corresponding amide and treated with TMSN₃/PPh₃/DEAD to yield the protected tetrazole.

### 5. Selective Formation of 1,5-disubstituted imidazoles as Angiotensin II Receptor Antagonists

Protection of N-1 position of imidazole by the trityl moiety allows the selective alkylation of ring at position N-3. Alkylation reagents can be varied according to designed targets. This allows introduction at position N-3 of several groups bearing desired (or modeling predicted) pharmacophoric groups. The alkylation reagent is the brominated phenyltetrazole derivative synthesized above. Alkylation with this reagent, followed by simultaneous deprotection of both protecting groups, provides the product as a TFA salt. This salt is neutralized with DIPEA, prior to selective protection of the tetrazole moiety.

### 6. Alkylation of N-1 trityl 4(5)Fmoc-alkyl-amine of Histamine

Alkylation of N-1 trityl 4(5)Fmoc-alkyl-amine of histamine is carried out with N-trityl-tetrazolyl-biphenyl-methyl-bromide in dichloromethane at room temperature (72 h).

### 7. Removal of Trityl Groups from Alkylated Derivative

Removal of the trityl groups is carried out with 50% TFA in dichloromethane solution in the presence of Et₃SiH as scavenger.

### 8. Tritylation of Tetrazole

Tritylation of tetrazole is carried out with an equimolar quantity of 2-chlorotritylchloride in the presence of base (diisopropylamine) in dichloromethane solution.

### 9. Deprotection of Fmoc Group

Deprotection of Fmoc group is carried out with 20% piperidine in DMF solution.

### SYNTHETIC EXAMPLES

Sartans with Clonidine-Like Activity

Guanylated Histamines

### Synthesis of N^{α}-guanyl-histamine 8

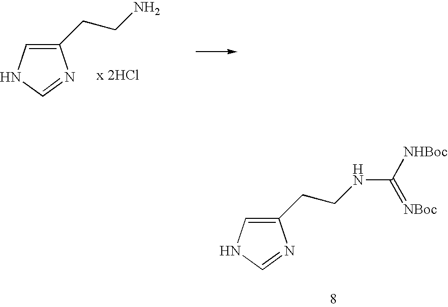

To a solution of histamine dihydrochloride (1.5 g, 8.15 mmol) in 1.25 mL H₂O, a solution of Na₂CO₃ (2.16 g, 20.38 mmol) in H₂O (6.2 mL) was added. The reaction mixture was left at ambient temperature for 30 min and the white precipitate was filtered. A solution of di-tert-butoxycarbonyl-S-methylisothiourea (2.6 g, 8.97 mmol) in THF (6.2 mL) was added to the filtrate. The resulting mixture was refluxed to 55° C. for 24 h. The progress was monitored by TLC to CHCl₃/MeOH (95:5). The reaction mixture was diluted with AcOEt and washed sequentially once with 5% aq. NaHCO₃ and twice with water. The mixture was dried over Na₂SO₄ and evaporated to dryness. The title product was obtained as a white solid (2.48 g, 86%) after Flash Column Chromatography (FCC) using as an eluant the system CHCl₃/MeOH (95:5).

ESI-MS (m/z): 253.93 (M+H−Boc), 153.98 (M+H−2Boc), 103.97 (Boc+2H).

¹H-NMR (CDCl₃): δ 7.57 (1H, s), 6.82 (1H, s), 3.65 (2H, q, J 6.0 Hz), 2.92 (2H, t, J 6.4 Hz), 1.53 (18H, d) ppm.

### Synthesis of N^{α}-guanyl-N^{τ}-trityl-histamine 9

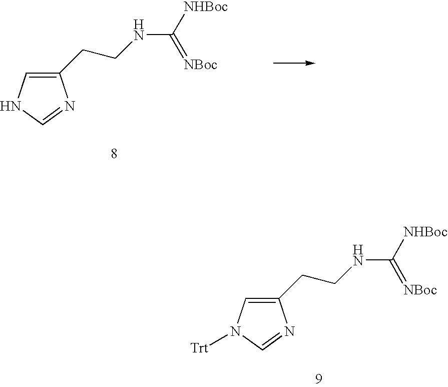

To an ice-cold solution of N^{α}-guanyl-histamine 8 (1 g, 2.83 mmol) in DCM (9 mL) were added sequentially DIPEA (1.22 mL, 7 mmol) and Trt-Cl (0.83 g, 2.97 mmol). After 1 h at 0° C., the reaction was completed as seen by TLC (CHCl₃/MeOH (97:3)). The reaction mixture was diluted with DCM and washed once with 5% aq. NaHCO₃ and twice with water. The mixture was dried over Na₂SO₄ and evaporated to dryness. To the oily residue was added Et₂O and placed in the refrigerator overnight. Filtration of the white precipitate results in the title compound 9 (1.43 g, 85%).

ESI-MS (m/z): 596.15 (M+H), 496.21 (M+H−Boc), 354.15 (M+2H−Trt), 154.12 (M+2H−Trt−2Boc), 103.97 (Boc+2H).

¹N-NMR (CDCl₃): δ 7.12-7.36 (17H, m), 6.65 (1H, s), 3.72 (2H, q, J 6.2 Hz), 2.79 (2H, t, J 6.8 Hz), 1.47 (18H, d) ppm.

### Synthesis of di-alkylated guanyl-histamine 10

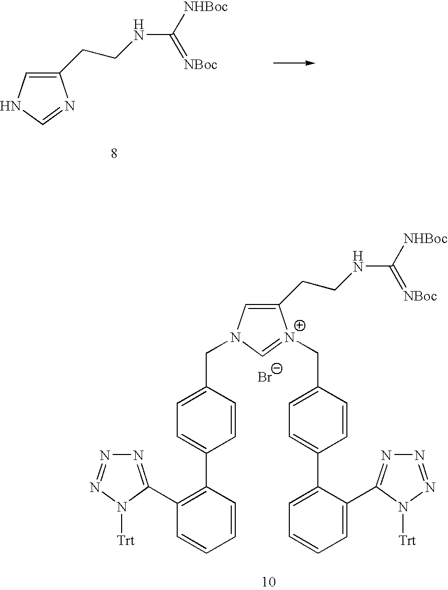

To a solution of 8 (0.2 g, 0.57 mmol) in DMF (2.5 mL), K₂CO₃ (0.2 g, 1.43 mmol) was added. The suspension was left to stir at ambient temperature for 30 min followed by the addition of compound 4 (0.82 g, 1.47 mmol). The resulting mixture was left for 24 h. The mixture was diluted with DCM and washed sequentially once with 5% aq. NaHCO₃ and twice with water. The organic layer was dried over Na₂SO₄ and evaporated to dryness. Et₂O was added and left overnight in the refrigerator. The resulting precipitate was filtered. Analytical HPLC (70% AcN/H₂O) of compound 10 showed it was of sufficient purity to use in the next step.

ESI-MS (m/z): 1388.53 (M+H), 1064.50 (M+H−Trt), 944.92 (M+H−Trt−2Boc), 821.38 (M+H−2Trt).

Hydroxymethylation of Histamine Compound 10

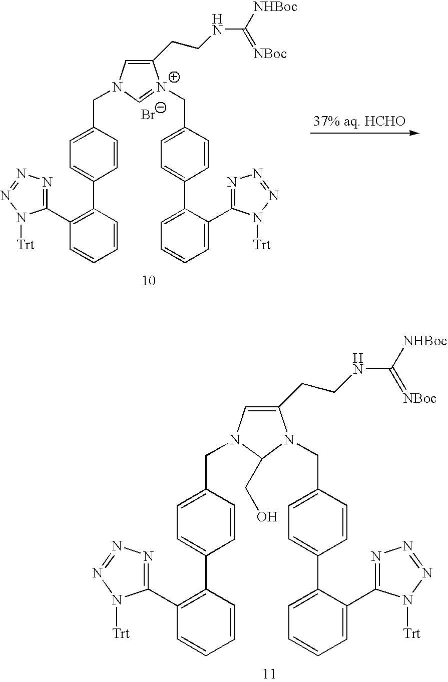

To a sealed tube containing 10 (0.2 g, 0.14 mmol) in DMF (2.5 mL) were added 37% aq. HCHO (165 μL) and DIPEA (128.5 μL). The resulting solution was left at 85° C. for 2 h. The reaction mixture was diluted with AcOEt, washed twice with a 5% aq. citric acid and twice with brine. The mixture was dried over Na₂SO₄ and evaporated under reduced pressure to yield compound 11 (0.12 g) as a yellow oil.

ESI-MS (m/z): 1339.60 (M+H), 1095.52 (M+H−Trt), 896.42 (M+H−Trt−2Boc), 851.40 (M+H−2Trt).

### Synthesis of Histamine Compound 12

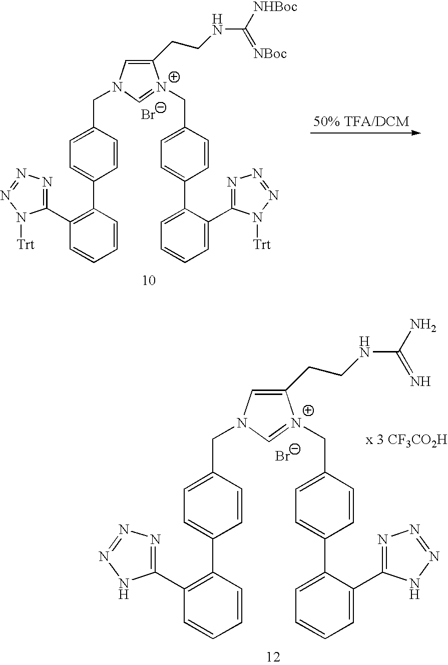

To a solution of 10 (0.5 g, 0.36 mmol), in DCM (4 mL) was added carefully TFA (4 mL) and a catalytic amount of Et₃SiH. The reaction mixture was left at ambient temperature for 5 h, evaporated to dryness, triturated with Et₂O and placed in the refrigerator overnight. The precipitate was filtered off and subjected to preparative HPLC using a preparative column, (Bondapak C18, 10 μm, 30×300 mm, flow rate 12 mL/min) with a gradient elution 30% AcN (0.08% TFA)/H₂O (0.08% TFA) to yield the desired product 12 (0.08 g, 22%) as a white solid after lyophilization.

ESI-MS (m/z): 623.70 (M+H−Br−TFA).

### Synthesis of Histamine Compound 13

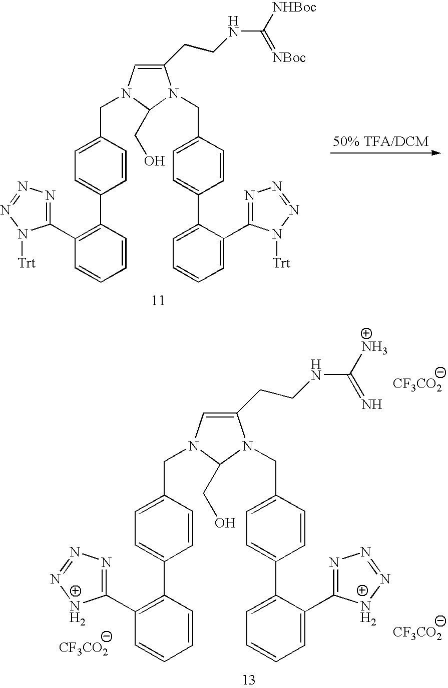

The deprotection procedure was identical to that of compound 10. Compound 11 (0.1 g, 0.074 mmol), was diluted to 1 mL of a solution 50% TFA in DCM. The final product (14 mg, 19%) was obtained as a white solid after preparative HPLC purification using a preparative column, (Bondapak C18, 10 μm, 30×300 mm, flow rate 12 mL/min) with a gradient elution 30% AcN (0.08% TFA)/H₂O (0.08% TFA) followed by lyophilization.

ESI-MS (m/z): 656.23 (M+H−Br−TFA).

### Synthesis of N(π)-alkylated guanylhistamine 15

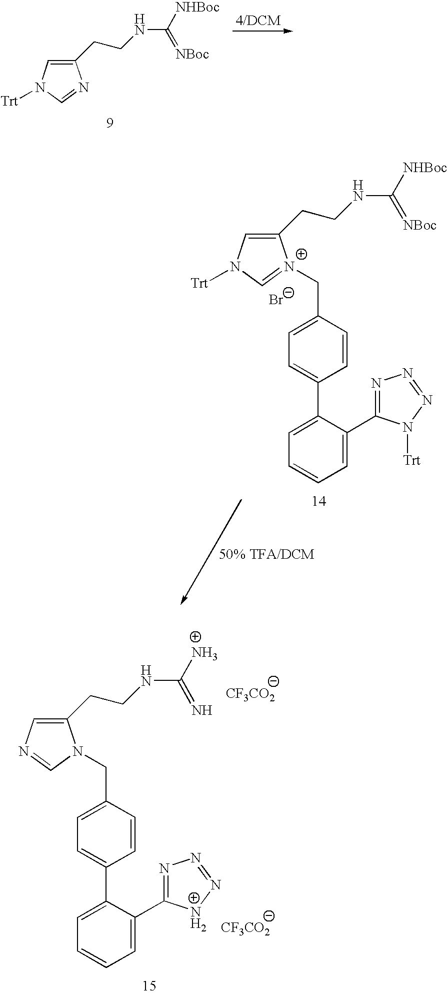

To a solution of 9 (0.32 g, 0.54 mmol) in DCM (1.5 mL), compound 4 (0.32 g, 0.57 mmol) was added. The reaction mixture was left at ambient temperature overnight, diluted with DCM and the organic layer washed once with a 5% aq. NaHCO₃ and twice with water. The mixture was dried over Na₂SO₄ and evaporated under vacuum. The oily residue was triturated with Et₂O and left overnight in the refrigerator. The Et₂O was decanted off and the intermediate 14 was of sufficient purity as shown by analytical HPLC. Intermediate 14 was dissolved in DCM (2 mL). TFA (2 mL) was added as well as a catalytic amount of Et₃SiH. The resulting solution was left at ambient temperature for 2 h, evaporated to dryness, triturated with Et₂O and left in the refrigerator. The Et₂O was decanted off and the oily residue was subjected to preparative HPLC σε 20% AcN/H₂O. The desired product 15 (73 mg, 22%) was obtained as a white solid after lyophilization.

ESI-MS (m/z): 389.23 (M+2H−CF₃CO₂⁻), 388.22 (M+H−CF₃CO₂⁻).

¹H-NMR (CDCl₃): δ 8.72 (1H, s), 7.65-7.10 (9H, m), 5.35 (2H, s), 3.10 (2H, t, J 6.4 Hz), 2.75 (2H, t, J 6.4 Hz) ppm.

### Synthesis of Compound 16

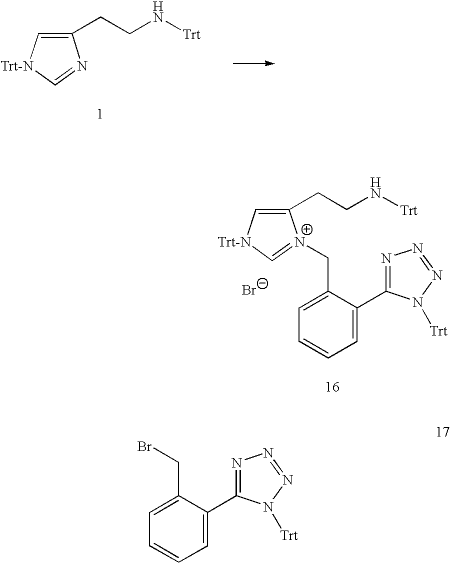

To a well stirred solution of 1 (0.6 g, 1 mmol) in DCM (3 mL) at ambient temperature, 17 (0.58 g, 1.2 mmol) was added and the resulting mixture left to stir for 48 h. The solvent was evaporated under vacuum and Et₂O was added to the oily residue. The resulting white precipitate was filtered and rinsed twice with Et₂O. The title compound was obtained 0.57 g (53%) as a white solid.

ESI-MS (m/z): 754.36 (M+H⁺−Trt−Br), 511.24 (M+H⁺−2Trt−Br)

### Synthesis of Compound 18

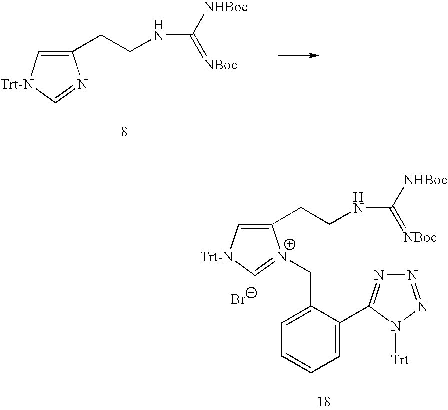

Same experimental procedure as in compound 14. The experiment was conducted on the following scale: N(τ)-trityl-guanylhistamine 8 (0.6 g, 1 mmol), DCM (3 mL), 17 (0.58 g, 1.2 mmol). Compound 18 was obtained 0.57 g (53%) as a white solid.

ES-MS (m/z): 997.11 (M+H⁺−Br), 998.22 (M+H₂−Br)⁺, 895.99 (M+H⁺−Boc−Br), 795.54 (M+H₂−2Boc−Br)⁺, 754.88 (M+H₂−Trt−Br)⁺, 653.76 (M+H₂−Trt−Boc−Br)⁺, 242.45 (Trt), 102.97 (Boc)

### Synthesis of Compound 20

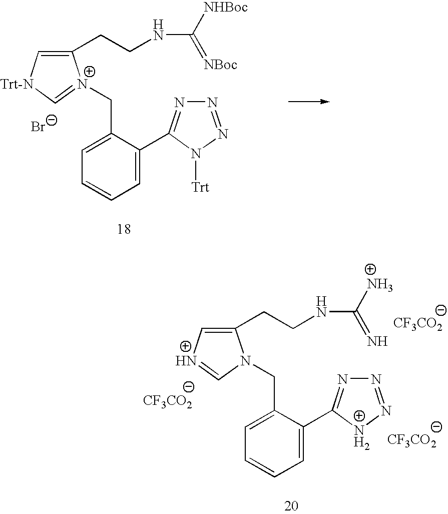

Same experimental procedure as in compound 15. The experiment was conducted on the following scale: 18 (0.5 g, 0.46 mmol), 50% TFA solution in DCM (5 mL) and catalytical amount of Et₃SiH. Compound 20 was obtained 0.14 g (58%) as a white solid.

ES-MS (m/z): 313.21 (M+H⁺−2TFA)

¹H-NMR (D₂O): δ 8.13-7.25 (5H, m), 5.56 (2H, s), 3.36 (2H, t, J 6.7 Hz), 2.74 (2H, t, J 6.7 Hz) ppm

Clonidine-Like Histamines

### Synthesis of N^{α}-trifluoroacetyl-N(τ)-trityl histamine

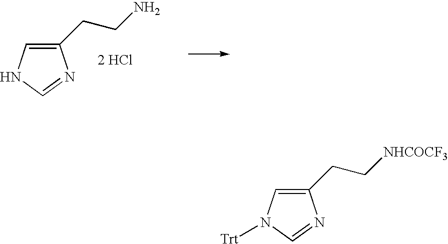

To an ice-cold suspension of histamine hydrochloride (1 g, 5.43 mmol) in MeOH (10 mL), was added carefully DIPEA (1.9 mL, 10.86 mmol). The resulting solution was left to stir at the above temperature for 10 min and then ethyl trifluoroacetate (0.65 mL, 5.43 mmol) was added. The reaction mixture was left at ambient temperature for 1 h. The solvent was evaporated under vacuum and the oily residue was diluted with DCM (10 mL). The reaction mixture was ice-cooled to 0° C. Et₃N (1.5 mL, 10.8 mmol) and trityl chloride (1.5 g, 5.4 mmol) were added subsequently. After 3 h at ambient temperature, the reaction mixture was diluted with DCM and washed once with 5% aq. NaHCO₃ and twice with water. The mixture was dried over Na₂SO₄ and evaporated to dryness. The crude residue was subjected to FCC at PhMe/AcOEt (7:3) to yield the title compound (2.29 g, 94%) as a white solid.

ESI-MS (m/z): 899.14 (2M+H), 450.12 (M+H).

¹H-NMR (CDCl₃): 8.40 (1H, br.s), 7.39 (1H, s), 7.36-7.33 (9H, m), 7.15-7.12 (6H, m), 6.62 (1H, s), 3.64 (2H, q, J 5.6 Hz), 2.76 (2H, d, J 6.4 Hz) ppm.

### Synthesis of N(τ)-trityl Histamine

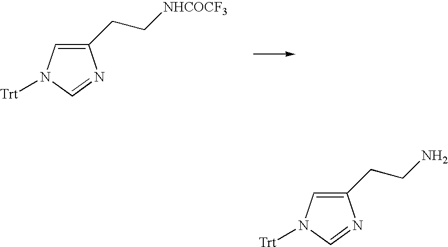

To a suspension of N^{α}-trifluoroacetyl-N(τ)-trityl histamine (1 g, 2.2 mmol) in MeOH (10 mL) was added a 4N aq. NaOH solution (1.5 mL). The resulting solution was left at ambient temperature for 4 h. The solvent was evaporated and the residue diluted with DCM. The organic layer was washed twice with brine, dried over Na₂SO₄ and evaporated to dryness to give the title compound (0.69 g, 89%) as a white oil.

ESI-MS (m/z): 707.86 (2M+H), 454.65 (M+H).

### Synthesis of N-(2-(1-trityl-1H-imidazol-4-yl)ethyl)-4,5-dihydro-1H-imidazol-2-amine

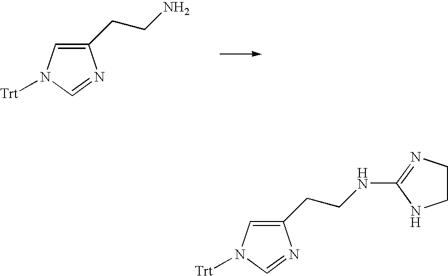

To a solution of N(τ)-trityl histamine (0.6 g, 1.7 mmol) in MeOH (10 mL) was added 2-methylthio-2-imidazoline hydroiodide (0.44 g, 1.8 mmol). The mixture was refluxed for 5 h. The reaction was left to cool to room temperature and then Et₃N (0.47 mL, 3.4 mmol) was added. The resulting mixture was left to stir for 30 min and then evaporated under vacuum. The oily residue was diluted with DCM. The organic layer was washed twice with water, dried over Na₂SO₄ and evaporated to dryness to yield the title compound.

ESI-MS (m/z): 422.50 (M+H), 179.49 (M+H−Trt).

Biological Activity

Purpose: To test the efficacy of various compounds with presumed α₂-AR agonist (clonidine) and AII receptor blockers (losartan) type properties.

Testing the α₂-agonist properties of E-P, H—P, H-Px

Experimental Protocol

Swiss Webster mice were anesthetized with 50 mg/kg pentobarbital and two lines were inserted, one in right iliac artery and the other in right iliac vein. Next morning the arterial line was connected to a blood pressure transducer and the mean BP was recorded with a Power lab/800 data acquisition system. The venous line was used for drug infusion. After one hour of measuring control blood pressure, 0.12 μg/kg of bolus norepinephrine was injected, and after allowing BP to return to baseline, 100 μg/kg of each compound (E-P, H—P, H-Px) as injected. After injection of the compound, BP was recorded for 30 minutes and a second bolus of NE was injected. In other mice, a known α₂-agonist UK 14304 (100 μg/kg) was injected instead of the compound and their actions were compared.

Results

We tested compounds E-P, H—P, H-Px, for an α2-AR agonist properties, using Swiss Webster mice. Results of each individual animal are shown in the Table 1. FIG. 1 presents the mean BP of each group during NE and compound injection. FIG. 2 presents the differences in BP after the first and second injection of NE. The first bolus of NE induced a hypertensive response ranging from 20-38 mmHg. This dose of NE was 10 times less than that used previously. Bolus injection of each compound produces a hypertensive response of 20-30 mmHg. This hypertensive effect was not followed by a subsequent decrease in BP. However, the hypertensive effect of NE 30 minutes after the injection of E-P, H—P, H-Px, was suppressed by 45%, 52% and 42% respectively. This was comparable with the effect of UK 14304, which suppressed the hypertensive effect of NE by 48%. In this aspect, all three compounds acted the same as UK 14304, but a higher dose is required for the compound E-P (300 mg/kg) to suppress NE effect, compared with 100 Ilg.kg for H—P and H-Px. UK 14304, (100 Ilg/kg) which was used as a control, decreased BP by 15 mmHg, but had no hypotensive effect.

TABLE 1
Testing Compounds for α₂ agonistic effect
Controls (UK injection)

| | Control | Bolus | BP after | Bolus | | |
|---|---|---|---|---|---|---|
| | BP | NE (0.12 | UK (100 | NE (0.12 | | |
| Mouse | (mmHg) | μg/kg) | μg/kg) | μg/kg) | Δ1 | Δ2 |
| 1 | 125.0 | 163.0 | 115.0 | 139.0 | 38.0 | 24.0 |
| 2 | 125.0 | 155.0 | 105.0 | 125.0 | 30.0 | 20.0 |
| 3 | 125.0 | 138.0 | 111.0 | 123.0 | 13.0 | 12.0 |
| 4 | 121.0 | 142.0 | 108.0 | 115.0 | 21.0 | 7.0 |
| Mean | 124.0 | 149.5 | 109.8 | 125.5 | 25.5 | 15.8 |
| SD | 2.00 | 11.56 | 4.27 | 9.98 | 10.85 | 7.68 |

Mean BP changes during NE and H-Px injection

| | Control | Bolus | BP after | Bolus | | |
|---|---|---|---|---|---|---|
| | BP | NE (0.12 | H-Px (100 | NE (0.12 | | |
| Mouse | (mmHg) | μg/kg) | μg/kg) | μg/kg) | Δ1 | Δ2 |
| 1 | 125.0 | 148.0 | 129.0 | 114.0 | 23.0 | 15* |
| 2 | 123.0 | 149.0 | 125.0 | 136.0 | 26.0 | 11.0 |
| 3 | 120.0 | 145.0 | 122.0 | 135.0 | 25.0 | 13.0 |
| Mean | 122.7 | 147.3 | 125.3 | 128.3 | 24.7 | 12.0 |
| SD | 2.52 | 2.08 | 3.51 | 12.42 | 1.53 | 1.41 |

Mean BP changes during NE and H-P injection

| | Control | Bolus | BP after | Bolus | | |
|---|---|---|---|---|---|---|
| | BP | NE (0.12 | H-P (100 | NE (0.12 | | |
| Mouse | (mmHg) | μg/kg) | μg/kg) | μg/kg) | Δ1 | Δ2 |
| 1 | 115.0 | 144.0 | 117.0 | 134.0 | 29.0 | 17.0 |
| 2 | 122.0 | 146.0 | 137.0 | 147.0 | 24.0 | 10.0 |
| 3 | 103.0 | 130.0 | 97.0 | 117.0 | 27.0 | 20.0 |
| Mean | 113.3 | 140.0 | 117.0 | 132.7 | 26.7 | 15.7 |
| SD | 9.61 | 8.72 | 20.00 | 15.04 | 2.52 | 5.13 |

Mean BP changes during NE and E-P injection

| | Control | Bolus | BP after | Bolus | | |
|---|---|---|---|---|---|---|
| | BP | NE (0.12 | E-P (100 | NE (0.12 | | |
| Mouse | (mmHg) | μg/kg) | μg/kg) | μg/kg) | Δ1 | Δ2 |
| 1 | 125.0 | 149.0 | 126.0 | 158.0 | 24.0 | 32.0 |
| 2 | 133.0 | 152.0 | 135.0 | 149.0 | 19.0 | 14.0 |
| Mean | 129.0 | 150.6 | 130.5 | 153.5 | 21.5 | 23.0 |
| SD | 5.66 | 2.12 | 6.36 | 6.36 | 3.54 | 12.73 |

Mean BP changes during NE and E-P injection

| | Control | Bolus | BP after | Bolus | | |
|---|---|---|---|---|---|---|
| | BP | NE (0.12 | E-P (300 | NE (0.12 | | |
| Mouse | (mmHg) | μg/kg) | μg/kg) | μg/kg) | Δ1 | Δ2 |
| 1 | 122.0 | 140.0 | 120.0 | 128.0 | 18.0 | 8.0 |
| 2 | 114.0 | 135.0 | 107.0 | 120.0 | 21.0 | 13.0 |
| 3 | 105.0 | 132.0 | 108.0 | 124.0 | 27.0 | 16.0 |
| Mean | 113.7 | 135.7 | 111.7 | 124.0 | 22.0 | 12.3 |
| SD | 8.50 | 4.04 | 7.23 | 4.00 | 4.58 | 4.04 |

Δ1 = BP changes between the first bolus of Norepinephrine and control BP
Δ2 = BP changes between second bolus of Norepinephrine and the new baseline BP after injection of compound.

Biological Evaluation of Compounds E-P, H—P, H-Px as Angiotensin II (AII) Receptor Blockers (ARBs)

Experimental Protocol

The hypotensive effect of these compounds as ARBs was evaluated in the anesthetized rabbit preparation made hypertensive by intravenous infusion of AII. In brief, adult normotensive male New Zealand White rabbits weighing between 2.5 and 3.5 kg were anesthetized by pentobarbitone (30 mg/kg), intubated and mechanically ventilated with 100% oxygen using a respirator for small animals (MD Industries, Mobile, Ala., USA). The tidal volume was 15 ml and the rate was adjusted to keep blood gases within normal range. Two polyethylene catheters were inserted, one in the carotid artery for continuous blood pressure monitoring via a transducer attached to a multichannel recorder (Nihon-Kohden, Model 6000, Japan) and the other one in the jugular vein for the administrations of solution made by diluting angiotensin II (ANGII) (Hypertension, CIBA) in 5% dextrose at final concentration of 5 μg/ml. Based on previous testing with this rabbit preparation submaximal angiotensin II-dependent hypertension was induced by infusing angiotensin via a syringe pump (Harvard Apparatus Pump 22, Harvard Apparatus, Natick, Mass., USA) at a constant rate of 0.2 ml/min (1 μg/min). Five minutes after the establishment of hypertension, two cumulative IV boluses of each compound were given via an ear vein 15 minutes apart. After the second bolus mean blood pressure was recorded for 30 minutes and the experiment was terminated. The compounds tested in these experiments included E-P, H—P and H-Px given as a first bolus of 300 ng/kg, followed after 15 minutes by a second bolus of 600 ng/kg. As positive controls, we used the known ARB's losartan, irbesartan and candesartan injected as a first bolus of 150 ng/kg, followed after 15 minutes by a second bolus of 300 ng/kg. In conclusion, compounds E-P, H—P, H-Px have α₂-AR agonist activity in mice, and also have AngII receptor blockers properties in rabbits. The novel compounds described in this invention, possess combined ANG II-blocking and SNS suppressing properties.

Results & Discussion

Compounds E-P, H—P, H-Px, were also tested for the AngII receptor blocker properties, by testing their ability to block the hypertensive effect of AngII, using adult normotensive male New Zealand white rabbits. Table 2 summarizes the antihypertensive effect and the degree of potency of the three novel compounds E-P, H—P, H-Px and the three known ARB's losartan, irbesartan and candesartan in a preparation of anesthetized rabbits made hypertensive by AII infusion. Values represent the mean blood pressure and are given as mean±S.D.

TABLE 2

| ARB's | | Baseline | Mean BP after | Mean BP after | Mean BP after | Cumulative % drop |
|---|---|---|---|---|---|---|
| Positive | | mean BP | AII infusion | 1^{st} bolus | 2^{nd} bolus | in mean BP after |
| Controls | N | (mmHg) | (1 μg/min) | (150 ng/kg) | (300 ng/kg) | the 2^{nd} bolus |
| Losartan | 3 | 117 ± 20 | 175 ± 7 | 148 ± 6 | 136 ± 3 | −22 ± 5 |
| Irbesartan | 4 | 133 ± 24 | 175 ± 13 | 141 ± 10 | 133 ± 11 | −24 ± 7 |
| Candesartan | 4 | 130 ± 18 | 197 ± 6 | 118 ± 6 | 117 ± 5 | −41 ± 3 |
| | | | Mean BP after | Mean BP after | Mean BP after | Cumulative % drop |
| Novel | | Baseline | AII infusion | 1^{st} bolus | 2^{nd} bolus | in mean BP after |
| Compounds | N | mean BP | (1 μg/min) | (300 ng/kg) | (600 ng/kg) | the 2^{nd} bolus |
| H-P | 5 | 135 ± 26 | 183 ± 17 | 166 ± 18 | 134 ± 10 | −26 ± 5 |
| H-Px | 4 | 117 ± 12 | 173 ± 20 | 148 ± 23 | 131 ± 15 | −24 ± 4 |
| E-P | 4 | 122 ± 9 | 161 ± 9 | 149 ± 7 | 140 ± 3 | −15 ± 4 |

In sharp contrast to peptide AII antagonists, which possess a partially agonistic effect when given intravenously, our novel compounds E-P, H—P, H-Px did not significantly affect mean BP after cumulative IV injections of 300-900 ng/kg in anesthetized normotensive rabbits.

The dose of compounds tested and the control ARBs given in our rabbit preparation is higher than that used in other experimental preparations i.e. mice, because we used a higher dose of AII infusion to substantially elevate mean BP to approximately 180 mmHg.

Various modifications and variations of the invention will be apparent to those skilled in the art without departing from the scope and spirit of the invention providing for molecules with double action i.e. Sympathetic System and Renin-Angiotensin System activities. Although the invention combining AT1 receptor activity and clonidine activity has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### REFERENCES

¹H-NMR Studies of [Sar¹] Angiotensin II Conformation by Nuclear Overhauser Effect Spectroscopy in the Rotating Frame (ROESY): Clustering of the Aromatic Rings in Dimethylsulfoxide, J. Matsoukas*, G. Bigam, N. Zhou and G. Moore, Peptides, 11, 359-366, (1990).
¹H-NMR Studies of Sarmesin and Des¹-Sarmesin Conformation in Dimethylsulfoxide by Nuclear Overhauser Effect (NOE) Enhancement Spectroscopy: Folding of the N- and C-terminal domains, J. Matsoukas*, R. Yamdagni, G. Moore, Peptides, 11, 367-374, (1990).
Tyrosinate Fluorescence Life Times for Oxytocin and Vasopressin in Receptor Simulating Environments: Relationship to Biological Activity and ¹H-NMR Data, R. J. Turner, J. M. Matsoukas, G. J. Moore, Bioch. Bioph. Res. Commun., 171, 996-1001, (1990).
Fluorescence Properties of Angiotensin II Analogues in Receptor-Simulating Environments: Relationship between Tyrosinate Fluorescence Lifetime and Biological Activity, R. J. Turner, J. M. Matsoukas and G. J. Moore, Biochimica et Biophysica Acta, 1065, 21-28, (1991).
Influence of Polyfluorination of the Phenylalanine Ring of Angiotensin II on Conformation and Biological Activity, P. R. Bovy, D. P. Getman, J. M. Matsoukas and G. J. Moore, Biochimica et Biophysica Acta, 1079, 23-28, (1991).
Synthesis and Biological Activities of Angiotensin II, Sarilesin and Sarmesin Analogues Containing Aze or Pip at Position 7: Conformational Properties of [Sar¹, Aze⁷] ANG II Determined by Nuclear Overhauser Effect (NOE) Enhancement Spectroscopy, J. Matsoukas, G. Agelis, J. Hondrelis, R. Yamdagni, Q. Wu, R. Ganter, D. Moore and G. J. Moore, J. Med. Chem., 36, 904-911, (1993).
Imidazole Based Non-Peptide Angiotensin II Receptor Antagonists: An Investigation of the Effect of the Orientation of the Imidazole Ring on Biological Activity, A Wahhab, J. R. Smith, R. C. Ganter, D. M. Moore, J. Hondrelis, J. Matsoukas and G. J. Moore, Drug Research, 43, 1157-1168, (1993).
Role of the NH₂-terminal Domain of Angiotensin II (ANG II) and [Sar1] Angiotensin II on Conformation and Activity, J. Matsoukas, J. Hondrelis, M. Keramida, T. Mavromoustakos, A. Makriyannis, R. Yamdagni, Q. Wu and G. J. Moore, J. Biol. Chem., 269, 5303-5311, (1994).
Novel Synthesis of Cyclic Amide-Linked Analogues of Angiotensins II and III, J. Matsoukas, J. Hondrelis, G. Agelis, K. Barlos, R. Ganter, D. Moore and G. J. Moore, J. Med. Chem., 37, 2958-2969, (1994).
Receptor Interactions of the Position 4 Side Chains of Angiotensin II Analogues: Importance of Aromatic Ring Quadrupole, G. J. Moore, R. C. Ganter, J. M. Matsoukas, J. Hondrelis, G. Agelis, K. Barlos, S. Wilkinson, J. Sandall and P. Fowler, J. Mol. Rec., 7, 251-256, (1994).
Design and Pharmacology of Peptide Mimetics, G. Moore, J. Smith, B. Baylis, J. Matsoukas, Advances in Pharmacology, 33, 91-141, (1995).
Differences in Backbone Structure between Angiotensin II Agonists and Type I Antagonists, J. Matsoukas, G. Agelis, A. Wahhab, J. Hondrelis, D. Panagiotopoulos, R. Yamdagni, Q. Wu, T. Mavromoustakos, H. Maia, R. Ganter and G. Moore, J. Med. Chem., 38, 4660-4669, (1995).
Advances in Antihypertensive Therapy: Non-Peptide Angiotensin II Receptor Antagonists as Potent Therapeutic Agents, J. Smith, A. Wahhab, J. Hondrelis, R. Ganter, D. Moore, J. M. Matsoukas and G. J. Moore, Letters in Peptide Science (LIPS), 1996, 3, 169-174 (Guest Editor of Special Issue).
Biological Activity of the Novel Cyclic Angiotensin II Analogues [Sar¹, Lys³, Glu⁵] ANG II, D. V. Vlahakos, J. M. Matsoukas, J. Ancans, G. J. Moore, E. K. Iliodromitis, K. P. Marathias and D. Th. Kremastinos, Letters in Peptide Science (LIPS), 1996, 3, 191-194 (Guest Editor of Special Issue).
Superimposition of Potent Non-Peptide AT₁ Receptor Antagonists with Angiotensin II, E. Theodoropoulou, Th. Mavromoustakos, D. Panagiotopoulos, J. Matsoukas and J. Smith, Letters in Peptide Science (LIPS), 1996, 3, 209-215 (Guest Editor of Special Issue).
An Effort to Understand the Molecular Basis of Hypertension Through the Study of Conformational Analysis of Losartan and Sarmesin Using a Combination of Nuclear Magnetic Resonance Spectroscopy and Theoretical Calculations, T. Mavromoustakos, A. Kolocouris, M. Zervou, P. Roumelioti, J. Matsoukas, R. Weisemann, J. Med. Chem. 1999, 42, 1714-1722.
Design and Synthesis of Thrombin Receptor-Derived Non-Peptide Mimetics Utilizing a Piperazine Scallold, K. Alexopoulos, P. Fatseas, E. Melissari, D. Vlahakos, J. Smith, T. Mavromoustakos, M. Saifeddine, G. Moore, M. Hollenberg, J. Matsoukas, Bioorganic and Medicinal Chemistry 1999, 7, 1033-1041.
The Bioactive Conformation of Angiotensin II. The Design and Synthesis of a Potent Angiotensin II Cyclic Analogue Confirms the Ring Cluster Receptor Conformation of the Hormone, J. Matsoukas, J. Ancans, T. Mavromoustakos, P. Roumelioti, D. V. Vlahakos, R. Yamdagni, Q. Wu, G. J. Moore, Bioorg. Med. Chem. 2000, 8, 1-10.
Structural Comparison Between Type I and Type II Antagonists: Possible Implications in the Drug Design of AT1 Antagonists, P. Roumelioti, T. Tselios et. al., Bioorg. And Med. Chem. Letters 2000, 10, 1-4.
In Hypertension: Pathophysiology, Diagnosis and Management, Sealey J. E., Laragh, J. H., Raven Press: New York 1990, 1, 1287.
In Hypertension Mechanisms; Harcourt Brace Jovanovich, Page I. H., New York 1987,347-470.
McAreavey D., Robertson, J. I. S., Drugs 1990, 40, 326.
Ondetti, M. A., Rubin, A., Cushman D. W., Science 1977, 196, 441.
Ondetti M. A., J. Med. Chem. 1981, 24, 355.
Timmermans P. B. M. W. M., Wong, P. C., Chiu A. T., Herblin W. F., Trends Pharmacol. Sci. 1991, 12, 55-62.
Duncia J. V., Chiu A. T., Carini D. J., Gregory, G. B., Johnson A. L., Price W. A., Wells G. J., Wong P. C., Calabrese J. C., Timmermans P. B. M. W. M., J. Med. Chem. 1990, 33, 1312.
Duncia J. V., Carini D. J., Chiu A. T., Johnson A. L., Price W. A., Wong P. C., Wexler R. R., Timmermans P. B. M. W. M., Med. Res. Rev. 1992, 12, 149.
In Hypertension: Pathophysiology, Diagnosis and Management, Timmermans P. B. M. W. M., Carine D. J., Chiu A. T., Duncia J. V., Price W. A., Wells G. J., Wong P. C., Wexter R. R., Johnson A. L., Raven Press: New York, 1990, 2351-2360.
Wexler R. R., Greenlee W. J., Irvin J. D., Goldberg M. R., Prendergast K., Smith R. D., Timmermans P. B. M. W. M., J. Med. Chem. 1996, 39, 625.
Wahhab A., Smith J., Ganter R. C., Moore D. M., Hondrelis J., Matsoukas J. M., Moore G. J., Arzneimittel-Forschung/Drug Res. 1993, 43, 1157.
Safety and efficacy of eprosartan, a new angiotensin II receptor blocker, N. H. Shusterman, American Heart Journal, 138, S238-S245, (1999).
The efficacy and tolerance of one or two daily doses of eprosartan in essential hypertension, Journal of Hypertension, 17, 129-136, (1999).
Pharmacokinetics of eprosartan in healthy subjects, patients with hypertension and special population, M. B. Bottorff, D. M. Tenero, Pharmacotherapy, 19, 73S-78S, (1999).
Review of eprosartan: A new angiotensin II receptor antagonist: Summary, D. A. Sica, Pharmacotherapy, 19, 108S-109S, (1999).
Treatment with the angiotensin II antagonist valsartan in patients with chronic renal failure and hypertension, J. Plum, B. Bunten, R. Nemeth, B. Grabensee, Nephrology Dialysis Transplantation, 14, 25-27, (1999).
Effect of valsartan on renal function in patients with hypertension and stable renal insufficiency, H. D. Faulhaber, J. F. Mann, G. Stein, L. Jansa, Current Therapeutic Research—Clinical and Experimental, 60, 170-183, (1999).
Influence of the angiotensin II antagonist valsartan on left ventricular hypertrophy in patients with essential hypertension, P. A. Thurmann, P. Kenedi, A. Schmidt, S. Harder, N. Rietbrock, Circulation, 98, 2037-2042, (1998).
Valsartan. A review of its pharmacology and therapeutic us in essential hypertension, Markham, K. L. Goa, Drugs, 54, 299-311, (1997).
Effects of the angiotensin II type 1 receptor blocker candesartan on endothelial function in patients with essential hypertension, L. Ghiadoni, A. Virdis, A. Magagna, S. Taddei, A. Salvetti, Hypertension, 35, 501-506, (2000).
Candesartan cilexetil: an angiotensin II receptor blocker, A. Stoukides, H. J. Mc Voy, A. F. Kaul, Annals of Pharmacology, 33, 1287-1298, (1999).
Candesartan: A new generation angiotensin II AT1 receptor blocker: Pharmacology, antihypertensive efficacy, renal function, and renoprotection, P. Morsing, Journal of the American Society of Nephrology, 10, S248-S254, (1999).
Candesartan cilexetil: A review of its use in essential hypertension, K. J. McClellan, K. L. Goa, Drugs, 56, 847-869, (1998).
Metabolites of the angiotensin II antagonist tasosartan: The importance of a second acidic group, J. W. Ellingboe, M. D. Collini, D. Quagliato, J. Med. Chem., 41, 4251-4260, (1998).
Effects of angiotensin antagonism with tasosartan on regional and systemic haemodynamics in hypertensive patients, Rheaume, P. H. Waib, Y. Lacourciere, J. Cleroux, Journal of Hypertension, 16, 2085-2089, (1998).
Tolerability profile of tasosartan, a long-acting angiotensin II AT1 receptor blocker, in the treatment of patients with essential hypertension, S. Oparil, A. Gradman, V. Papademetriou, M. Weber, Current Therapeutic Research—Clinical and Experimental, 58, 930-943, (1997).
Blood pressure control: A review on irbesartan, B. Waeber, European Heart Journal, Supplement, 2, B2-B7, (2000).
Safety of irbesartan in the treatment of mild to moderate systemic hypertension, T. A. Simon, R. T. Gelarden, S. A. Freitag, K. B. Kassler-Taub, R. Davies, The American Journal of Cardiology, 82, 179-182, (1998).
Irbesartan. A review of its pharmacodynamic and pharmacokinetic properties and therapeutic use in the management of hypertension, J. C. Gillis, A. Markham, Drugs, 54, 885-902, (1997).
Clinical overview of irbesartan: Expanding the therapeutic window in hypertension, J. Man in't Veld, Journal of Hypertension, Supplement, 15, S27-S33, (1997).
Efficacy and safety of telmisartan, a selective AT1 receptor antagonist, compared with enalapril in elderly patients with primary hypertension, E. Karlberg, L. E. Lins, K. Hermansson, Journal of Hypertension, 17, 293-302, (1999).
The efficacy and safety of telmisartan compared to enalapril in patients with severe hypertension, J. M. Neutel, D. H. G. Smith, P. A. Reilly, International Journal of Clinical Practice, 53, 175-178, (1999).
Hypotensive effects of the angiotensin II antagonist telmisartan in conscious chronically-instrumented transgenic rats, J. C. A. Van Meel, N. Redemann, R. M. Haigh, Arzn.-Forsch./Drug Research, 46, 755-759, (1996).
An angiotensin converting enzyme inhibitor to identify and treat vasoconstrictor and volume factors in hypertensive patients, H. Gavras, H. R. Brunner, J. H. Laragh, J. E. Sealey, I. Gavras, R. A. Vukovich, N. Engl. J. Med. 291, 817-821, (1974).
Angiotensin II inhibition: treatment of congestive cardiac failure in a high-renin hypertension, H. Gavras, A. Flesas, T. J. Ryan, H. R. Brunner, D. P. Faxon, I. Gavras, JAMA, 238, 880-882, (1997).
Suppressing sympathetic activation in congestive heart failure. A. J. Manolis, C. Olympios, M. Sifaki, S. Handanis, M. Bresnaban, I. Gavras, H. Gavras, Hypertension, 26, 719-724, (1995).
Combined sympathetic suppression and angiotensin converting enzyme inhibition in congestive heart failure, A. J. Manolis, C. Olympios, M. Sifaki, S. Handanis, D. Cokkinos, M. Bresnahan, I. Gavras, H. Gavras, Hypertension, 29(part 2), 525-530, (1997).
Effects of specific inhibitor of the vascular action of vasopressin in humans, H. Gavras, A. B. Ribeiro, O. Kohlmann, M. Saragoca, R. A. Mulinari, I. Gavras, Hypertension, 6 (Suppl 1), 156-160, (1984).
Synthesis and some pharmacologic properties of five novel V1 or Z1/V2 antagonists of AVP, B. Lammek, Y. X. Wang, I. Derdowska, R. franco, H. Gavras, Peptides, 10, 1109-1112, (1989).
Oral administration of DuP753, a specific angiotensin II receptor antagonist, to normal male volunteers, Y. Christen, B. Waeber, J. Nussberger, M. Porchet, R. m. Borland, R. J. Lee, K. Maggon, L. Shu, P. B. M. W. M. Timmermans, H. R. Brunnear, Circulation, 83, 1333-1342, (1991).

1. A compound of formula I, 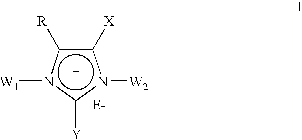
wherein
R is H, halogen;
Y is H, CH₂O-alkyl, CH₂S-alkyl, CH₂OH, CH₂SH, CH₂-halogen, COOH, halogen or CHO;
X is (CH₂)ₙR₁, wherein R₁ is —NH₂, —NHR′, —NH—C(═NH)NH₂, —NH—C(═NR′)—NHR″ or 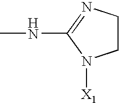
wherein X₁ is —COOCMe₃, —COMe, —COEt, COPh, trityl, halotrityl or benzyl;
n is 1 to 10;
R′ and R″ are each independently alkyl, cycloalkyl, alkyl-cycloalkyl, or an amino or guanadino nitrogen protecting group, PG₁, or R′ and R″ are linked to form a cyclic group;
W₁ and W₂ are each independently —(CH₂)ₘ—K—Z—Z₁, where m is 1 to 5;
K is biphenyl or monophenyl;
Z is tetrazolyl or COO—;
Z₁ is H, trityl, halotrityl, CH₂(Ph), COOH, COO-alkyl or CH(Ph)₂, wherein each Ph group is optionally substituted by one or more halogens; and
E is an anion;
or a pharmaceutically acceptable salt thereof.

2. A compound according to claim 1 wherein X is (CH₂)ₙ—NH—C(═NH)NH₂, (CH₂)ₙ—NH₂ or (CH₂)ₙ—R₁, wherein R₁ is 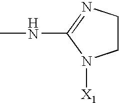
and wherein X₁ is —COOCMe₃, —COMe, —COEt, COPh, trityl, halotrityl or benzyl; n is 1 to 5;
and E is a halo ion.

3-5. (canceled)

6. A compound according to claim 1 wherein W₁═W₂.

7. A compound according to claim 1 wherein W₁ is

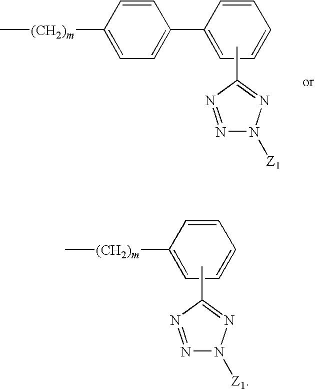

8-9. (canceled)

10. A compound according to claim 1 wherein m is 1; Y is H, CH₂OH, CH₂OMe, CH₂OEt, CH₂SH, CH₂SMe, halogen or CH₂Set; R is H, Cl, Br, F, I; Z₁ is H, trityl, halotrityl, dibenzyl or benzyl; X is (CH₂)ₙ—NH—C(═NH)NH₂, (CH₂)ₙ—NH₂, or (CH₂)ₙ—R₁, wherein R₁ is 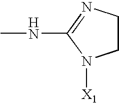
and wherein X₁ is —COOCMe₃, —COMe, —COEt, COPh, trityl, halotrityl or benzyl.

11-15. (canceled)

16. A compound according to claim 1, wherein said compound is of formula E, 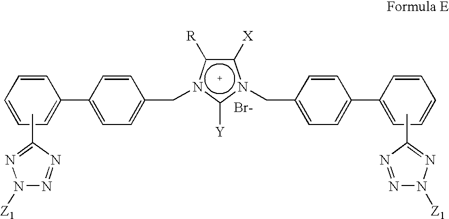
wherein:
X is (CH₂)ₙ—NH—C(═NH)NH₂, (CH₂)ₙ—NH₂ or (CH₂)ₙ—R₁where 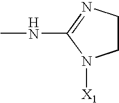
wherein X₁ is —COOCMe₃, —COMe, —COEt, COPh, trityl, halotrityl or benzyl;
n is 1 to 5;
Y is H, CH₂O-alkyl, CH₂S-alkyl, CH₂OH, CH₂SH or CHO;
R is H or halogen; and
Z₁ is H, trityl, 2-chlorotrityl or benzyl or formula F, 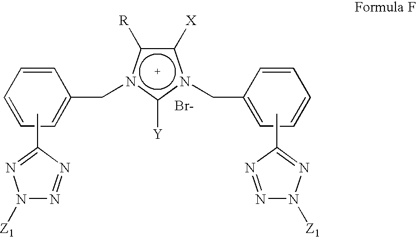
wherein:
X is (CH₂)ₙ—NH—C(═NH)NH₂, (CH₂)ₙ—NH₂ or (CH₂)ₙ—R₁ where 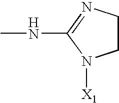
wherein X₁ is —COOCMe₃, —COMe, —COEt, COPh, trityl, halotrityl or benzyl;
n is 1 to 5;
Y is H, CH₂O-alkyl, CH₂S-alkyl, CH₂OH, CH₂SH or CHO;
R is H or halogen; and
Z₁ is H, trityl, 2-chlorotrityl or benzyl.

17-18. (canceled)

19. A compound of formula IIa or IIb, 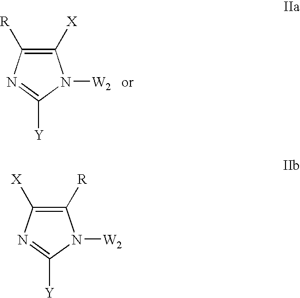
wherein
R is H, halogen;
Y is H, CH₂O-alkyl, CH₂S-alkyl, CH₂OH, CH₂SH, CH₂-halogen, COOH, halogen or CHO;
X is (CH₂)ₙR₁, wherein R₁ is —NH₂, —NH—C(═NH)NH₂, —NH—C(═NR′)—NHR″ or 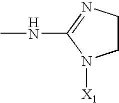
wherein X₁ is —COOCMe₃, —COMe, —COEt, COPh, trityl, halotrityl or benzyl;
n is 1 to 10;
R′ and R″ are each independently alkyl, cycloalkyl, alkyl-cycloalkyl, or an amino or guanadino nitrogen protecting group, PG₁, or R′ and R″ are linked to form a cyclic group;
W₂ is —(CH₂)ₘ—K—Z—Z₁, where m is 1 to 5;
K is biphenyl or monophenyl;
Z is tetrazolyl or COO—; and
Z₁ is H, trityl, halotrityl, CH₂(Ph), COOH, COO-alkyl or CH(Ph)₂, wherein each Ph group is optionally substituted by one or more halogens;
or a pharmaceutically acceptable salt thereof.

20-21. (canceled)

22. A compound according to claim 19 wherein W₂ is

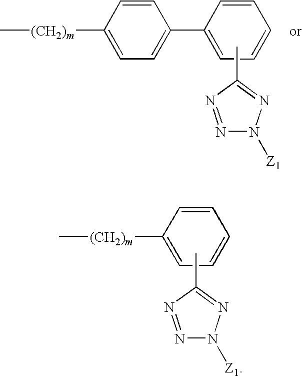

23. (canceled)

24. A compound according to claim 19 wherein m is 1;
R is H, Cl, Br, F or I;
Z₁ is H, trityl, halotrityl, dibenzyl or benzyl;
X is (CH₂)ₙ—NH—C(═NH)NH₂, (CH₂)—NH₂, or (CH₂)ₙR₁, where R₁ is 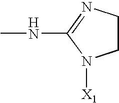
wherein X₁ is —COOCMe₃ —COMe —COEt, COPh, trityl, halotrityl or benzyl.

25-29. (canceled)

30. A compound according to claim 19 wherein said compound is of formula A, 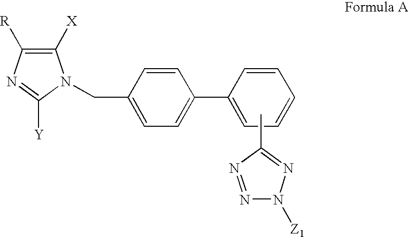
wherein
Y is H, CH₂O-alkyl, CH₂S-alkyl, CH₂OH, CH₂SH or CHO;
R═H, halogen;
X is (CH₂)ₙ—NH₂;
n is 1 to 5; and
Z₁ is H, trityl, 2-chlorotrityl or benzyl; or
formula B, 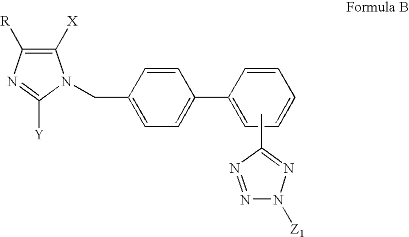
wherein
Y is H, CH₂O-alkyl, CH₂S-alkyl, CH₂OH CH₂SH or CHO;
R═H, halogen;
X is (CH₂)ₙ—NH—C(═NH)NH₂;
n is 1 to 5; and
Z₁ is H, trityl, 2-chlorotrityl or benzyl, or formula C, 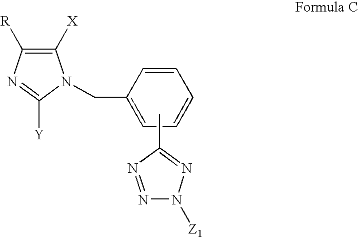
wherein
Y is H, CH₂O-alkyl, CH₂S-alkyl, CH₂OH CH₂SH or CHO;
R═H, halogen;
X is (CH₂)ₙ—NH₂;
n is 1 to 5; and
Z₁ is H, trityl, 2-chlorotrityl or benzyl, or formula D, 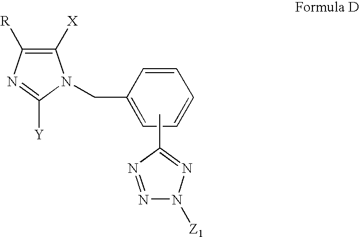
wherein
Y is H, CH₂O-alkyl, CH₂S-alkyl, CH₂OH CH₂SH or CHO;
R═H, halogen;
X is (CH₂)ₙ—NH—C(═NH)NH₂;
n is 1 to 5; and
Z₁ is H, trityl, 2-chlorotrityl or benzyl, or formula G, 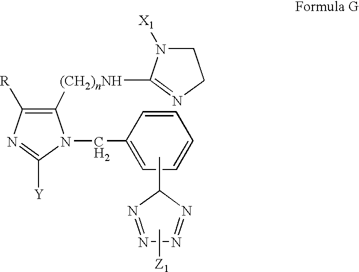
wherein
Y is H, CH₂O-alkyl, CH₂S-alkyl, CH₂OH, CH₂SH or CHO;
R═H, halogen;
X₁ is —COOCMe₃, —COMe, —COEt, COPh, trityl, halotrityl or benzyl;
n is 1 to 5; and
Z₁ is H, trityl, 2-chlorotrityl or benzyl, or 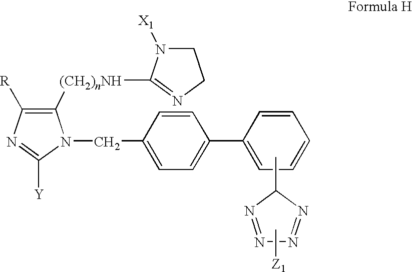
wherein
Y is H, CH₂O-alkyl, CH₂S-alkyl, CH₂OH CH₂SH or CHO;
R═H, halogen;
X₁ is —COOCMe₃, —COMe, —COEt, COPh, trityl, halotrityl or benzyl;
n is 1 to 5; and
Z₁ is H, trityl, 2-chlorotrityl or benzyl.

31-35. (canceled)

36. A compound according to claim 19 wherein Y is selected from H, —CH₂OH, CH₂SH, CH₂OMe, CH₂SMe, CH₂OEt, CH₂Set, Z₁ is H or trityl, and R is H or Cl.

37. (canceled)

38. A compound according to claim 19 which is selected from:

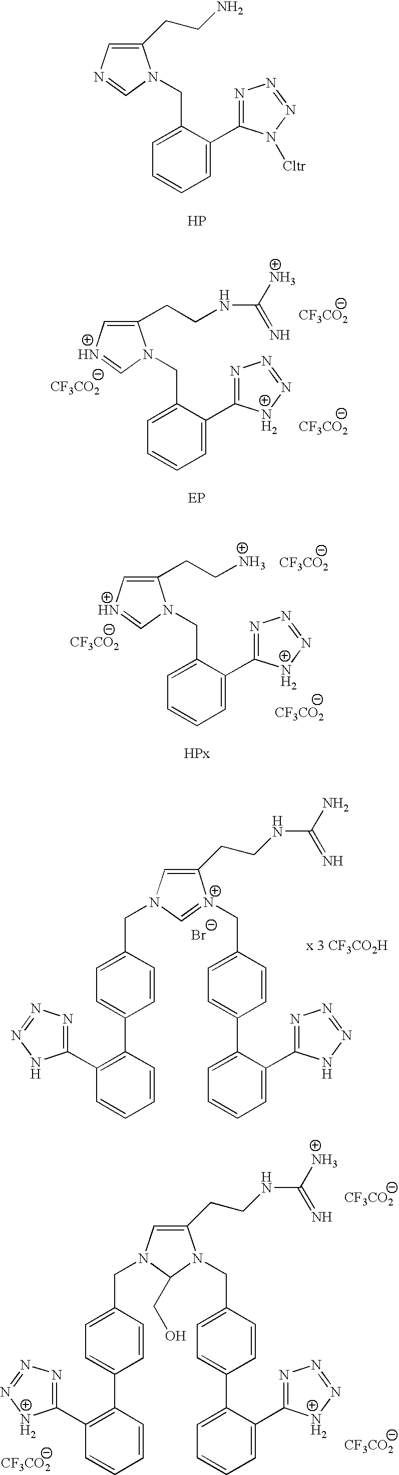

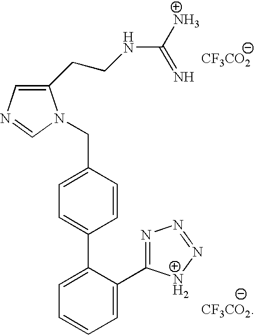

39. (canceled)

40. A pharmaceutical composition comprising a compound as defined in claim 1, or a pharmaceutically acceptable salt thereof, admixed with a pharmaceutically acceptable diluent, excipient or carrier.

41-43. (canceled)

44. A method of treating hypertension or a cardiovascular disorder in a subject, said method comprising administering to the subject a therapeutically effective amount of a compound according to claim 1, or a pharmaceutically acceptable salt thereof.

45. A method according to claim 44 wherein the compound is administered transdermally.

46. A process for preparing a compound of formula IIa as defined in claim 19, wherein X is (CH₂)ₙ—NH₂, said process comprising the steps of: 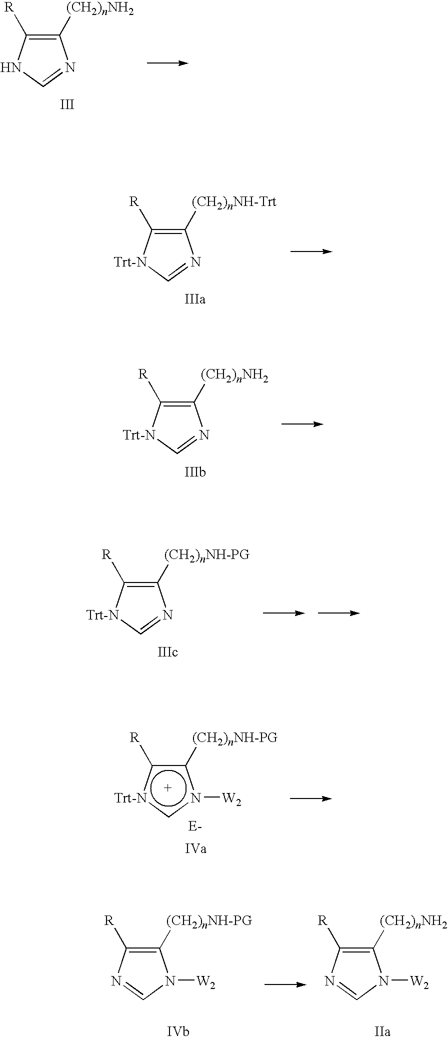
(i) reacting a compound of formula III with trityl chloride to form a compound of formula IIIa;
(ii) reacting said compound of formula Ma with TFA to form a compound of formula IIIb;
(iii) protecting the free NH₂ group of said compound of formula IIIb with a protecting group, PG, to form a compound of formula IIIc;
(iv) reacting said compound of formula Mc with Br—(CH₂)ₙ—K—Z—Z₁ to form a compound of formula IVa;
(v) converting said compound of formula IVa to a compound of formula IVb; and
(vi) removing protecting group PG to form a compound of formula IIa.

47-49. (canceled)

50. A process for preparing a compound of formula IIa as defined in claim 19, wherein X is (CH₂)ₙ—NH—C(═NH)NH₂, said process comprising the steps of: 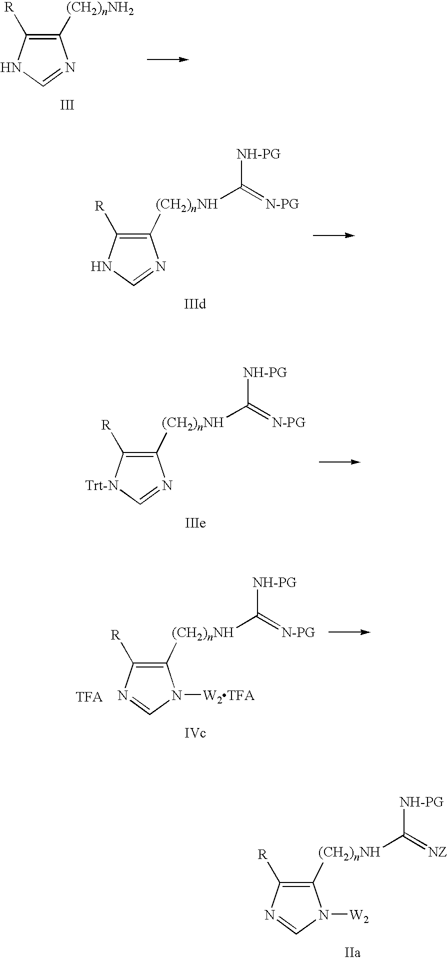
(i) reacting a compound of formula III with PG-HN—C(SMe)=N—PG to form a compound of formula IIId;
(ii) reacting said compound of formula IIId with trityl chloride to form a compound of formula IIIe;
(iii) reacting said compound of formula IIIe with Br—(CH₂)ₙ—K—Z—Z₁ to form a compound of formula IVc;
(iv) converting said compound of formula IVc to a compound of formula IIa; and
(vi) converting said compound of formula IIa to a compound of formula IIa.

51. A process for preparing a compound of formula I as defined in claim 1, wherein X is (CH₂)ₙ—NH₂, said process comprising the steps of: 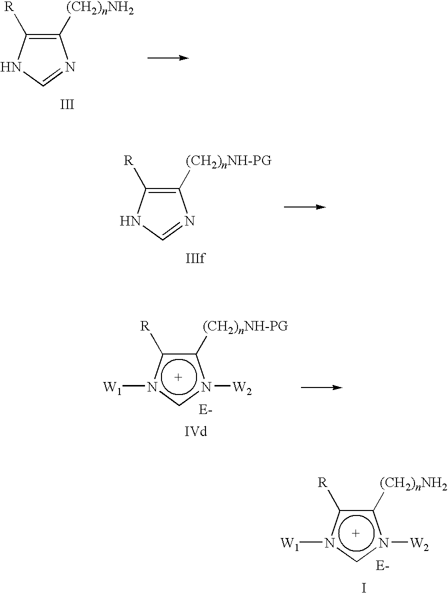
(i) protecting the free NH₂ group of a compound of formula III with a protecting group, PG, to form a compound of formula IIIf;
(ii) reacting said compound of formula IIIf with Br—(CH₂)ₙ—K—Z—Z₁ to form a compound of formula IVd; and
(iii) removing protecting group PG from said compound of formula IVd to form a compound of formula I.

52-53. (canceled)

54. A process for preparing a compound of formula I as defined in claim 1, wherein X is (CH₂)ₙ—NH—C(═NH)NH₂, said process comprising the steps of: 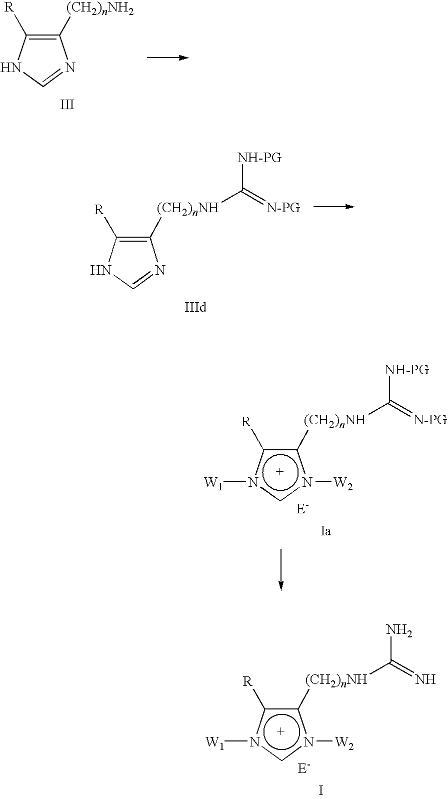
(i) reacting a compound of formula III with PG-HN—C(SMe)=N—PG to form a compound of formula IIId;
(ii) reacting said compound of formula IIId with Br—(CH₂)ₙ—K—Z—Z₁ to form a compound of formula Ia;
(iii) removing protecting groups PG from said compound of formula Ia to form a compound of formula I.

55-56. (canceled)
